(19) United States
(12) Patent Application Publication
(10) Pub. No.: US 2026/0049268 A1
(43) Pub. Date: Feb. 19, 2026

(54) IN VITRO CELL CULTURE MUCUS SYSTEMS

(71) Applicant: The University of North Carolina at Chapel Hill, Chapel Hill, NC (US)

(72) Inventors: Nancy L. Allbritton, Chapel Hill, NC (US); Yuli Wang, Cary, NC (US)

(73) Assignee: The University of North Carolina at Chapel Hill, Chapel Hill, NC (US)

(21) Appl. No.: 19/275,031

(22) Filed: Jul. 21, 2025

Related U.S. Application Data
(63) Continuation of application No. 17/284,766, now Pat. No. 12,410,391.
(60) Provisional application No. 62/768,259, filed on Nov. 16, 2018.

(51) Int. Cl. C12M 1/12; C12M 1/06; C12N 5/071

(52) U.S. Cl. CPC C12M 25/04; C12M 27/02; C12N 5/068

(57) ABSTRACT

This presently disclosed subject matter relates to an in vitro cell culture comprising a cell monolayer comprising mucus producing cells and a mucus layer, and methods of making and using the same. The methods including culturing mucus producing cells on a cell support structure under conditions to establish a mucus layer on the luminal side of the cell monolayer, thereby producing a live cell construct comprising a cell monolayer comprising mucus producing cells and a mucus layer. The mucus layer can be substantially impenetrable to micro-objects, and have a thickness of about 1 micron to about 1 cm.

### CROSS REFERENCE TO RELATED APPLICATIONS

This application is a continuation of, claims the benefit of and priority to, previously filed U.S. patent application Ser. No. 17/284,766, filed Apr. 12, 2021, which is a 35 U.S.C. § 371 national phase entry of International Application No. PCT/US2019/061743, filed Nov. 15, 2019, which claims the benefit of and priority to U.S. Provisional Patent Application Ser. No. 62/768,259, filed Nov. 16, 2018, the disclosures of each of which are incorporated herein by reference in their entireties.

### STATEMENT OF GOVERNMENT SUPPORT

This invention was made with government support under grant number DK109559 awarded by National Institutes of Health. The government has certain rights to this invention.

### TECHNICAL FIELD

The present disclosure relates to an in vitro cell culture comprising a cell monolayer comprising mucus producing cells and a mucus layer, and methods of making and using the same.

### BACKGROUND

The luminal surfaces of the small and large intestine are protected by a thick blanket of mucus. The small intestine produces a 50 to 450 μm single layer of mucus, while the large intestinal mucus coating is composed of two structurally distinct layers.¹ The 400 to 800 μm outer mucosal layer is loosely packed, providing a hospitable environment for bacteria to inhabit, while the 100 to 300 μm inner mucosal layer remains impenetrable to bacteria and firmly attached to the epithelium.^{2,3} In both organs, mucus is continuously generated and secreted by goblet cells lining the intestinal epithelium. The mucus layer above the cells is estimated to be replaced on the order of a few hours.⁴ Mucin 2 (Muc2) is the most abundant gel-forming component of intestinal mucus.⁵ A mucus layer is essential to sustain the homeostasis of the colonic mucosa in vivo. The main functions of the mucus layer are to serve as a barrier to protect the underlying epithelium from pathogen invasion, as well as to keep the lining of the colon lubricated and moist due to the high concentration of water (about 97%) present in mucus.^{6, 7} The mucus layer also acts as a barrier to hinder diffusion of molecules derived from food or bacteria and their metabolites, and traps molecules such as secretory IgA (sIgA) and anti-bacterial peptides secreted by intestine.⁸

In vitro intestinal epithelium models provide a valuable tool to study the highly complicated intestinal epithelium system under a controlled manner. Adenocarcinoma cell lines including Caco-2 and HT-29 are widely used as in vitro models of the intestinal epithelium, but they do not produce dense mucus layers.⁹ HT29-MTX, a stable homogenous subpopulation of HT-20 derived after treatment with methotrexate, is often used as a mucus-secreting cell model.¹⁰ However, these tumor cells do not possess normal signaling pathways, physiologic mucus secretion, or appropriate responses to external stimuli. In recent years, breakthroughs in intestinal stem cell biology permit building in vitro models based on primary intestinal epithelial stem cells.¹¹ The organoid culture model, for the first time, realized the in vitro expansion and lineage manipulation of adult intestinal epithelial stem cells, and the generated “mini-gut” organoids possess the diversity of cell lineages found in in vivo epithelium, including mucus-secreting goblet cells.¹¹⁻¹⁴ The cells are polarized so that their apical surface faces to the enclosed lumen and their basal sides are attached to Matrigel® or other ECM components.¹⁵ Mucus, however, is secreted into and accumulated within the organoids' lumen which is surrounded by a layer of cells as well as a dense hydrogel such as Matrigel® or collagen. This mucus secretion is not readily quantified or manipulated nor does the mucus act as a barrier to external stimuli.

In attempts to expose the luminal surface, monolayer models have been built by culturing the intestinal epithelial stem cells (e.g. from isolated crypts, or dissociated organoid fragments) on porous membranes coated with either a thick or thin layer of extracellular matrix.¹⁶⁻²³ The cells proliferated in the presence of growth factors (Wnt-3A, R-spondin and noggin), and differentiated in the absence of growth factors to form continuous monolayers possessing a physiologic transepithelial electrical resistance (TEER). Monolayers possess a unique advantage over organoids with their open luminal surface, which allows easy access to the apical epithelium to assay the impact, absorption or metabolism of food components, microorganisms, bioactive metabolites, drugs, and toxic compounds. The monolayer model has been used to study IgA transcytosis,¹⁶ co-culture with bacteria,^{17, 18} iron transport,¹⁹ hormone secretion,¹⁹ co-culture with macrophages,²⁰ co-culture with myofibroblasts and enteric neurons,²¹ and cytokine secretion.^{20, 21} In all of the above reported monolayer models of primary cells, the cells were in a submerged culture system, i.e. aqueous medium was placed into the basal and luminal reservoirs. Traces of mucus were identified by staining with Muc2 antibody.¹⁸ The mucus thickness was measured by overlaying the cells with fluorescent microbeads and measuring the distance between the cells and beads which are too large to penetrate the mucus.¹⁶ However, the mucus layer was not continuous,¹⁸ nor thick enough to separate the microbeads from the epithelium.¹⁶ Thus the beads appear to be in contact with the apical epithelial surface within the resolution of the microscopic images. However, Muc2 was detected in the supernatant above the cells suggesting that some amount of Muc2 was synthesized and secreted but not in sufficient quantities or densities to recreate a mucus layer.²⁴

Thus, a dense mucus layer that is substantially microbead or bacterial-impenetrable with a controllable thickness has not yet been achieved in any in vitro intestinal epithelium models constructed from primary intestinal epithelial cells. Such an in vitro model is needed to reflect in vivo conditions.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are methods of producing a live cell construct comprising a cell monolayer comprising mucus producing cells and a mucus layer, comprising culturing stem cells that are capable of differentiating into mucus producing cells (e.g., intestinal epithelial stem cells, basal stem cells, induced pluripotent stem cells and the like) on an upper surface of a cell support structure having both an upper surface and a lower surface until at least a portion of the upper surface of the cell support structure is covered by the stem cells, and culturing the stem cells further to produce a cell monolayer comprising mucus producing cells (e.g., goblet cells GC) and other cell types (OC, including enterocytes, enteroendocrine cells, Paneth cells, stem cells, etc.), the cell monolayer having a basal side and a luminal (apical) side, wherein the mucus producing cells of the cell monolayer establish a mucus layer on the luminal side of the cell monolayer, thereby producing a live cell construct comprising a cell monolayer comprising mucus producing cells and a mucus layer. The mucus layer can be substantially or completely impenetrable to micro-objects. In some aspects, the thickness of the mucus layer can be about 1 micron to about 1 cm. In some embodiments, the thickness of the mucus layer can be about 30 microns to about 1 cm. The ratio of GC to OC can in some embodiments range from about 0.1% to about 99.9%.

In some embodiments, a basal reservoir is present below the basal side of the cell monolayer comprising mucus producing cells and a luminal reservoir is present above the luminal side of the cell monolayer comprising mucus producing cells, and the basal reservoir and the luminal reservoir each comprise a liquid medium; the method further comprising: removing the liquid medium in the luminal reservoir to produce an air-liquid interface at the luminal side of the cell monolayer that comprises mucus producing cells, and/or adjusting the volume of the liquid medium in the luminal reservoir to a depth in a range of about 0.001 mm to about 10 mm, optionally about 0.001 mm to about 1 mm, above the luminal side of the cell monolayer (wherein the mucus layer is (or develops) between the liquid medium and the cell monolayer). In some embodiments, the methods can further comprise positioning an impermeable physical barrier and/or a partially permeable physical barrier on or above the luminal side of the cell monolayer comprising mucus producing cells. In some aspects, the impermeable physical barrier and/or the partially permeable physical barrier is in direct contact with the luminal side of the cell monolayer comprising mucus producing cells and/or the mucus layer produced by the mucus producing cells of the cell monolayer. The liquid medium can be between the impermeable physical barrier and/or the partially permeable physical barrier and the luminal side of the cell monolayer comprising mucus producing cells and/or the mucus layer and the depth of the liquid medium is in a range of about 0.001 mm to about 10 mm, optionally about 0.001 mm to about 1 mm. In some embodiments, the liquid medium comprises a hormone, a chemical additive, a food additive, bacterial metabolite, and/or a hypertonic salt solution, wherein the hormone, the chemical additive, the food additive, the bacterial metabolite, and/or the hypertonic salt solution. In some embodiments, the stem cells are epithelial stem cells, intestinal epithelial stem cells, basal stem cells, induced pluripotent stem cells, respiratory stem cells, gastric stem cells, nasal stem cells, reproductive tract cells (cervix, vagina, uterus), urethra cells, olfactory cells, mouth cells, tongue cells, and/or conjunctiva cells. In some embodiments, the stem cells are intestinal epithelial stem cells. The mucus layer can be substantially impenetrable to beads or microorganisms in a size range from about 1 to about 100 microns.

In some embodiments, a force is applied parallel to the surface of the cell monolayer. In some embodiments, the force comprises application of a surface tension force or application of a mechanical force. In some embodiments, the mechanical force is a stir bar, a semi-solid material moving parallel to the cell surface, and/or circulation of a slurry on the top of cell surface.

In some embodiments, provided herein is a live cell construct comprising a cell monolayer comprising mucus producing cells and a mucus layer produced by the methods disclosed herein. In some embodiments, the mucus layer comprises a basal side and a luminal side, wherein the basal side is below and adjacent to the mucus producing cells.

In some embodiments, provided herein are live cell constructs comprising a cell monolayer comprising mucus producing cells and a mucus layer, wherein the mucus layer is substantially impenetrable to micro-objectives. The mucus layer can comprise a thickness of about 1 micron to about 1 cm. The mucus layer can be impenetrable to micro-objects in a size range from about 1 micron to about 100 microns. In some embodiments, the mucus layer can comprise a basal side and a luminal side, wherein the basal side is below and adjacent to the mucus producing cells.

In some embodiments, provided herein are methods of determining the ability of an organism, a drug, or a particle to traverse (penetrate) a mucus layer of a cell monolayer, the method comprising contacting the luminal side of the mucus layer of the live cell construct disclosed herein with the organism, drug, or particle, and measuring the distance that the organism, drug, or particle moves into the mucus layer, thereby determining the ability of the organism, drug or particle to traverse (penetrate) the mucus layer of the cell monolayer of the live cell construct.

In some embodiments, provided herein are methods of studying and evaluating an organism's ability to infect a cell monolayer comprising a mucus layer, comprising contacting the luminal side of the mucus layer of the live cell construct disclosed herein with the organism, and determining if the organism traverses the mucus layer and contacts the cell monolayer of the live cell construct, wherein when the organism is determined to traverse the mucus layer and contact the cell monolayer, the organism is determined to be able to infect the cell monolayer comprising a mucus layer.

In some embodiments, provided herein are methods of evaluating the effectiveness of a drug to prevent or reduce infection by an organism, comprising contacting the luminal side of the mucus layer of the live cell construct disclosed herein with the organism, contacting the luminal side of the mucus layer of the live cell construct with the drug, and determining whether the organism penetrates the mucus layer of the cell monolayer and/or infects one or more the cells of the cell monolayer of the live cell construct, wherein the drug is determined to be effective for preventing or reducing infection if the organism does not penetrate the mucus layer and/or infect one or more cells of the cell monolayer of the live cell construct and is determined to not be effective if the organism penetrates the mucus layer and/or infect one or more cells of the cell monolayer of the live cell construct as compared to a control (i.e., contacted with the organism but no drug). Contacting the organism with the luminal side of the mucus layer of the live cell construct can be prior to, concurrent with, or after contacting the drug with the luminal side of the mucus layer of the live cell construct.

In some embodiments, provided herein are methods of evaluating an immunological response of a cell comprising a mucus layer to invasion by an organism, contact by a particle, and or contact by a chemical/compound, comprising contacting the luminal side of the mucus layer of the live cell construct disclosed herein with the organism, particle and or chemical/compound, and assaying cells of the cell monolayer of the live cell construct for the production of a marker associated with an immune response (e.g., a cytokine, a chemokine, a hormone, a neurotransmitter, and/or a antimicrobial peptide), thereby evaluating the immunological response of the cell monolayer of the live cell construct to contact by the organism, particle and or chemical/compound. The organism can be a bacterium, a virus, a fungus, protozoan, and/or a helminth.

In some embodiments, disclosed herein are methods of evaluating mucus misregulation in an in vitro cell system, comprising studying the mucus layer of the live cell construct disclosed herein, wherein the stem cells that are cultured are from a subject having a disease associated with mucus misregulation and/or the stem cells that are cultured are from a healthy subject and are gene edited to recapitulate stem cells from a disease associated with mucus misregulation; and studying comprises evaluating the mucus layer of the live cell construct for thickness, composition, viscosity, degree of penetration by micro-objects, ability of microorganisms to infect, and/or responsiveness to drugs; thereby evaluating mucus misregulation in an in vitro cell system. In some embodiments, a disease associated with mucus misregulation is inflammatory bowel disease, constipation, cystic fibrosis irritable bowel syndrome, leaky gut syndrome, bacterial overgrowth syndromes, celiac disease, lactose intolerance, excessive gas syndromes, diarrheal diseases, and/or polyps appendicitis.

The foregoing and other objects and aspects of the present disclosure are explained in detail in the specification set forth below.

Embodiments of the presently disclosed subject matter having been stated hereinabove, and which are achieved in whole or in part by the presently disclosed subject matter, other embodiments will become evident as the description proceeds when taken in combination with the accompanying Examples as best described hereinbelow.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A through 1E provide schematics of systems and methods according to the present disclosure for generating an in vitro mucus layer. FIG. 1A provides an air-liquid interface (ALI) culture. The apical side is exposed to air and the liquid or medium has been removed allowing mucus to accumulate in a compacted form. FIG. 1B illustrates a modified ALI culture. The volume of liquid is controlled at the apical side allowing mucus to accumulate in a compacted form and having increased hydration. FIG. 1C illustrates an example of use of a scaffold in place of a porous membrane. FIG. 1D provides an example of use of a partially permeable physical barrier (separator) to confine accumulated mucus. FIG. 1E illustrates an example of use of an impermeable physical barrier (blocker) to control mucus accumulation.

FIGS. 2A through 2E provide schematic illustrations of the creation of a compact mucus layer removal of liquid from the apical epithelial side (ALI culture), and resulting data. FIG. 2A is a schematic showing the submerged and ALI culture formats, including stem cells (SC), mucus producing goblet cells (GC), other non-mucus producing cells (OC), and mucins (or diluted mucins) (M). FIG. 2B shows immunofluorescence staining of cross sections through paraffin-embedded monolayers (Muc2 and nuclei labelled). Arrows demarcate goblet cells. A: apical side; B: basal side. The lower panels (i, ii and iii) show higher magnification regions as marked by the corresponding dotted line boxes in the upper two panels. FIG. 2C shows apical surface topography of human colonic monolayers inspected by SEM. Top panel: submerged culture. Bottom panel: ALI culture. In FIGS. 2D and 2E the mucus layer was overlaid with 1 μm red fluorescent beads (FIG. 2D) or GFP-expressing E. coli (FIG. 2E) for visualization by confocal microscopy. The nuclei of intestinal cells were stained with Hoechst 33342. The dashed line shows the boundary between the mucus and microbeads or E. coli.

FIGS. 3A through 3G show that a hydrated mucus layer separates bacteria or microbeads from epithelium incubated with vasoactive intestinal peptide (VIP). FIG. 3A is a schematic showing the culture format. VIP facilitates the water movement into the lumen or luminal side LS which hydrates the mucus layer. FIG. 3B is a graphical depiction of data showing the concentration-dependent water secretion by VIP after 24 h of incubation. FIG. 3C is an image of the hydrated mucus layer lifted off the epithelium by forceps. FIG. 3D includes representative side-view confocal micrographs showing tissues with bacteria-separating mucus accumulation at 0, 2, 4 and 6 days, respectively. GFP-expressing E coli and nuclei are labelled. FIG. 3E is a plot of mucus thickness versus duration of ALI. FIG. 3F is a representative side-view confocal micrograph showing the mucus layer separated 1 μm red fluorescent beads from epithelium. FIG. 3G shows images of apical surface topography of the epithelium inspected by SEM. The mucus layer was partially removed to reveal the epithelium (dashed line). The upper right panel shows bacteria (rod-shaped structures) above the mucus layer and absent from the epithelial surface.

FIGS. 4A through 4F show the effect of C. difficile toxin A on human colonic epithelium in the absence or presence of the VIP-enhanced mucus layer. FIG. 4A is a schematic illustration showing the cell culture systems. FIGS. 4B and 4C show permeability (FIG. 4B) and IL-8 secretion (FIG. 4C) of epithelium after 4-h exposure to toxin A. FIG. 4D shows confocal microscopic images showing F-actin architecture in the apical membrane (top panel) and ZO-1 staining tight junctions (bottom panel). FIGS. 4E and 4F show permeability (FIG. 4E) and IL-8 secretion (FIG. 4F) of epithelium after 8-h exposure to toxin A. Unpaired t test: * P<0.05; ** P<0.005; # not statistically significant. N=3 samples per condition. Scale bar=20 μm.

FIGS. 5A through 5E show cytokine production after a 24 h co-culture of GFP-expressing E. coli (GFP EC), epithelium and PBMCs in the absence or presence of the VIP-enhanced mucus layer. FIG. 5A is a schematic illustration showing the co-culture setup. The graphical depictions of FIGS. 5B through 5E show the production of cytokines at the basal side for 24 h: (FIG. 5B) IL-8, (FIG. 5C) TNF-α, (FIG. 5D) IL-6 and (FIG. 5E) IL-1β. Unpaired t test: ** P<0.005; # not statistically significant. N=3 samples per condition.

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

Unless otherwise defined, all technical and scientific terms used herein have the same meaning as commonly understood by one of ordinary skill in the art to which this presently disclosed subject matter belongs. The terminology used in the description of the presently disclosed subject matter herein is for the purpose of describing particular embodiments only and is not intended to be limiting of the presently disclosed subject matter.

All publications, patent applications, patents and other references cited herein are incorporated by reference in their entireties for the teachings relevant to the sentence and/or paragraph in which the reference is presented.

Unless the context indicates otherwise, it is specifically intended that the various features of the presently disclosed subject matter described herein can be used in any combination. Moreover, the presently disclosed subject matter also contemplates that in some embodiments of the presently disclosed subject matter, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

Like numbers refer to like elements throughout. In the figures, the thickness of certain lines, layers, components, elements or features can be exaggerated for clarity. Where used, broken lines illustrate optional features or operations unless specified otherwise.

As used in the description of the presently disclosed subject matter and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value can include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

It will be understood that when an element is referred to as being “on,” “attached” to, “connected” to, “coupled” with, “contacting,” etc., another element, it can be directly on, attached to, connected to, coupled with and/or contacting the other element or intervening elements can also be present. In contrast, when an element is referred to as being, for example, “directly on,” “directly attached” to, “directly connected” to, “directly coupled” with or “directly contacting” another element, there are no intervening elements present. It will also be appreciated by those of skill in the art that references to a structure or feature that is disposed “adjacent” another feature can have portions that overlap or underlie the adjacent feature.

Spatially relative terms, such as “under,” “below,” “lower,” “over,” “upper” and the like, can be used herein for ease of description to describe an element's or feature's relationship to another element(s) or feature(s) as illustrated in the figures.

It will be understood that, although the terms first, second, etc., can be used herein to describe various elements, components, regions, layers and/or sections, these elements, components, regions, layers and/or sections should not be limited by these terms. Rather, these terms are only used to distinguish one element, component, region, layer and/or section, from another element, component, region, layer and/or section. Thus, a first element, component, region, layer or section discussed herein could be termed a second element, component, region, layer or section without departing from the teachings of the presently disclosed subject matter. The sequence of operations (or steps) is not limited to the order presented in the claims or figures unless specifically indicated otherwise.

The term “comprise,” “comprises” and “comprising” as used herein, specify the presence of the stated features, integers, steps, operations, elements, and/or components, but do not preclude the presence or addition of one or more other features, integers, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

As used herein, the terms “increase,” “increasing,” “increased,” “enhance,” “enhanced,” “enhancing,” and “enhancement” (and grammatical variations thereof) describe an elevation of at least about 5%, 10%, 15%, 20%, 25%, 50%, 75%, 100%, 150%, 200%, 300%, 400%, 500% or more as compared to a control.

As used herein, the terms “reduce,” “reduced,” “reducing,” “reduction,” “diminish,” and “decrease” (and grammatical variations thereof), describe, for example, a decrease of at least about 5%, 10%, 15%, 20%, 25%, 35%, 50%, 75%, 80%, 85%, 90%, 95%, 97%, 98%, 99%, or 100% as compared to a control. In particular embodiments, the reduction can result in no or essentially no (i.e., an insignificant amount, e.g., less than about 10% or even 5%) detectable activity or amount.

As used herein, the term “micro-objects” means an object having a dimension of about 0.1-100 μm, which can be used to determine the penetrability of a mucus layer. Examples of micro-objects include, but are not limited to, microorganisms (e.g., bacteria) and micro-particles (e.g., beads).

The instant disclosure describes the discovery of methods of generating live cell constructs comprising cell monolayers comprising mucus producing cells and a mucus layer that recapitulates the in vivo mucus layer present in, for example, the intestinal tract and/or the reproductive tract of human and animal subjects. A thick, in vivo-like mucus barrier can be important for successful recreation of the gut environment for microbiota, as well as can serve as an in vitro tool to investigate mucosal drug and particle delivery. While mucus production has been observed previously in in vitro cell systems, these systems have not been able to produce mucus in sufficient quantities or densities to recreate a mucus layer that mimics the mucus layer found in in vivo settings. Without being limited to any particular theory, it is postulated herein that the mucus in these previous in vitro cell systems can be rapidly diluted after it is secreted from goblet cells in submerged culture systems, and therefore is unable to accumulate and form a dense layer (substantially impenetrable to microorganisms and micro-particles). As described herein, the presently disclosed subject matter overcomes these limitations.

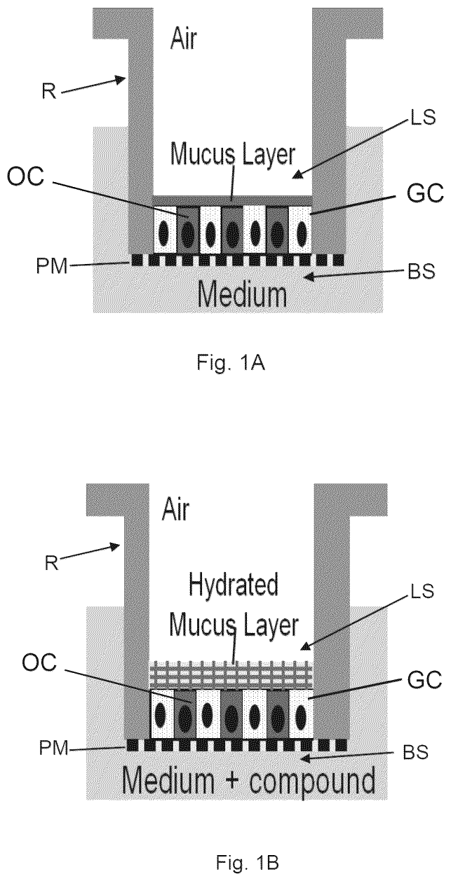

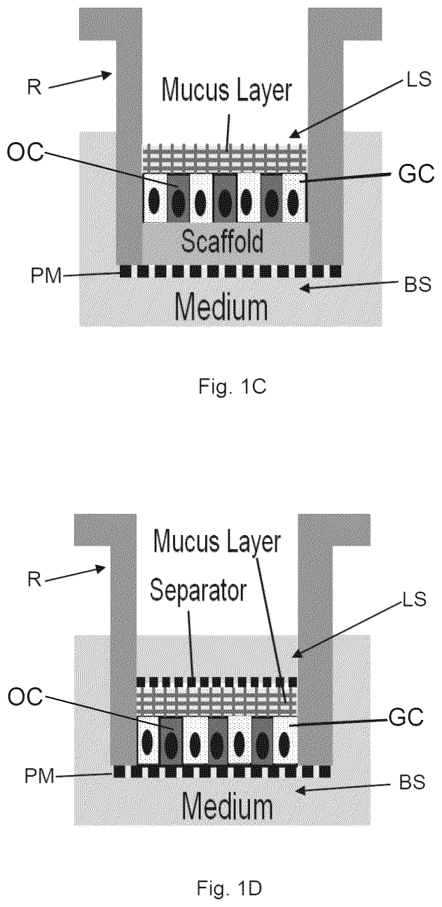

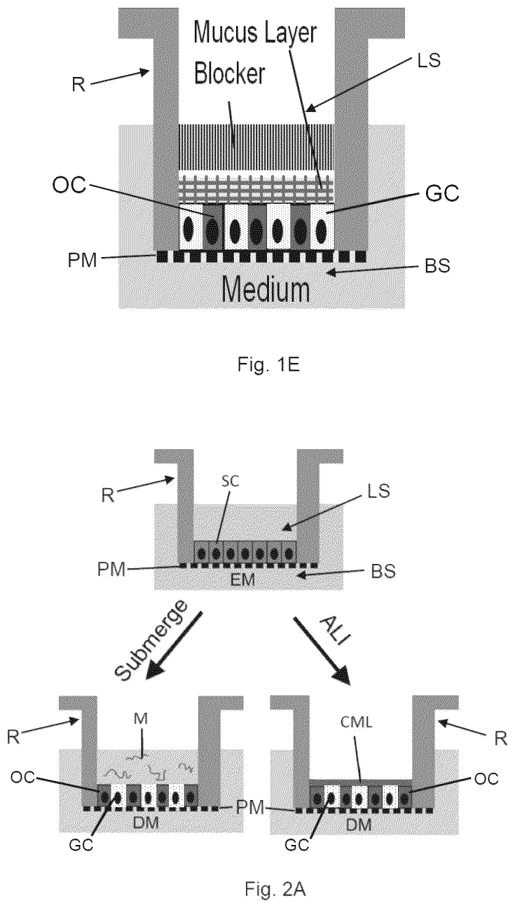

In some embodiments, and as shown in FIGS. 1A through 1E, the present disclosure provides a live cell construct in a reservoir R comprising a cell layer that contains a combination or mixture of mucus-secreting goblet cells GC and other non-mucus producing cells OC, e.g. enterocytes, enteroendocrine cells, Paneth cells, stem cells, etc., the cells having a luminal side LS or apical side, as well as a basal side BS to the cells with a porous matrix (PM)/scaffold/base below the cells; and methods that facilitate mucus accumulation above the apical/luminal side LS of the cells to form a dense, in vivo-like mucus layer. The ratio of GC to OC can in some embodiments range from about 0.1% to about 99.9%. Thus, a cell monolayer in a live cell construct as disclosed herein can in some aspects comprise a mixture of mucus producing cells and non-mucus producing cells.

To generate a cell layer that contains mucus-secreting cells, epithelial stem cells are expanded on the surface of a porous membrane. The membrane can be coated with a thin (e.g., about 1 to 1,000 nanometers) extracellular matrix (ECM) or other chemical or with a thick (e.g., about 1 to about 100,000 micrometers) hydrogel scaffolding material (ECM or synthetic hydrogel) to support the cells. The cells are grown in media until a portion of the surface is covered with the cells. The stem cells SC can be allowed to spontaneously differentiate as they begin to cover the surface and/or consume nutrients in the adjacent medium. Alternatively, the cells can be switched to a different media that encourages the formation of mucus producing cells (e.g., goblet cells (GC)) and other cell types (OC) or chemical additives can be incorporated into the media to induce differentiation (e.g., butyrate, bone morphogenetic protein (BMP), gamma secretase inhibitors (e.g. DAPT, LY411575, dibenzazepine), etc.

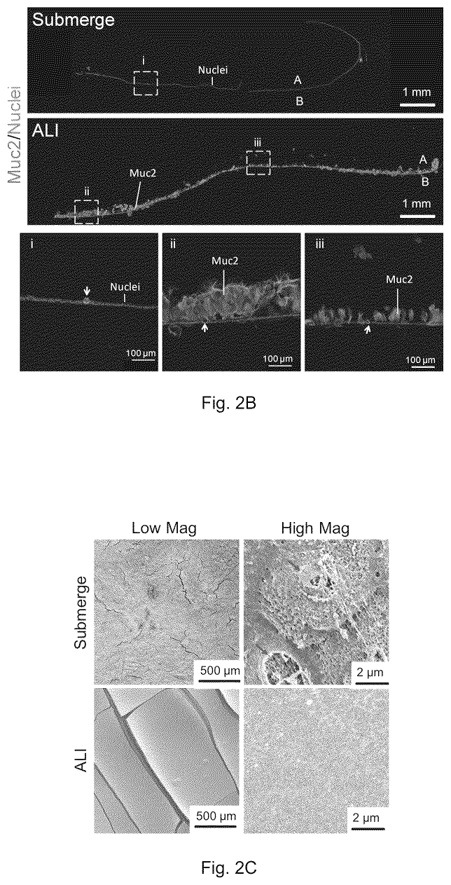

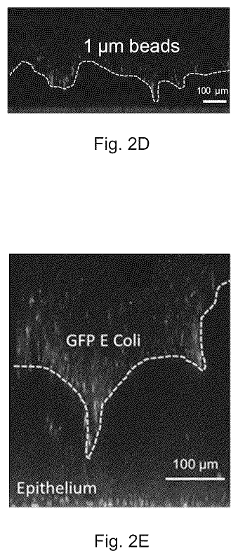

A mucus layer can then be established on the apical cell side/luminal cell side LS using different methods as illustrated in FIGS. 2A through 2E. For example, as shown in FIG. 2A, an air-liquid interface (ALI) culture can be prepared in which liquid or medium is removed from the apical reservoir, or luminal side LS, or luminal reservoir. The ALI conditions permit mucus accumulation above the mucus producing goblet cells GC and/or OC (forming the epithelium) along the apical surface on the luminal side LS (as opposed to the basal side BS) in a dense compacted layer, or compacted mucus layer CML. In some embodiments, an ALI condition can allow the accumulation of a dense or concentrated mucus layer CML by (a) minimizing dilution of mucus that is secreted, (b) increasing forces on the cell surface due to surface tension or other effects (that promote mucus production and/or secretion), and/or (c) increasing local medium osmolality above the cells, etc. Movement of water through the mucus producing goblet cells GC) due to evaporation at their surface can also stimulate mucous production. See, Example 1.

As another example, a modified ALI culture can be prepared as shown in FIG. 1B. This approach provides control of the height of the liquid medium on the apical/luminal side LS of the mucus producing cell monolayer, so that the mucus accumulated above the cell monolayer is dense, hydrated, and in vivo-like, i.e. a hydrated mucus layer. A liquid or medium depth in the range of about 0.001 mm to about 10 mm can be provided and maintained above the apical or luminal cell side LS so that the mucus, which accumulates over time is in a more hydrated state relative to that of ALI. The volume of liquid/medium in the luminal side LS of the reservoir R can be controlled to produce mucus with a range of mucus densities and compactness depending on the experimental needs.

In some embodiments, one or more materials or substances can be added to the apical surface or luminal side of the cells to assist in maintaining an aqueous film above the apical cell surface and can also be used to assist in programming or maintaining the desired liquid height over time. For example, a semi-liquid mass (e.g. hydrogels), a gas-impermeable membrane, a gas permeable membrane, and hygroscopic materials (honey, glycerin, sugar, nylon, ABS (acrylonitrile/butadiene/styrene), polycarbonate, cellulose, and poly (methyl methacrylate)) can be placed on the apical surface. Chemical reagents, hormones, food metabolites, bacterial products and other compounds can also be added to the culture system to assist in programming the desired fluid height and consequently also the mucus thickness and density. See, Example 2, where a hormone is added to the basal medium to promoting luminal water secretion.

Turning now to FIG. 1C, in addition to the use of a porous membrane PM upon which a cell monolayer, e.g. differentiated epithelial cells, can be cultured, in some aspects such cells can be cultured or supported on a porous or nonporous scaffold. Examples of scaffolds that support a cell monolayer include, but are not limited to, hydrogel (natural and synthetic), porous material, nonporous material, plastic, ceramics, etc. Other examples include inorganic materials or a composite of organic and inorganic materials. Examples of inorganic materials suitable for supports include, but are not limited to, glass, hydroxyapatite, Bioglass such as 45S5 Bioglass, calcium phosphate, silicon, silicon oxide, titanium oxide, gold, aluminum oxide, etc. Where not inherently porous, these materials can be made porous by a variety of methods, including but not limited to, sintering, etching, leaching, lithography, etc. For example, a porous mesh of silicon and gold can be fabricated by lithography/etching. Such scaffolds can be supported on or placed adjacent to porous membrane PM.

FIG. 1D shows the use of a separator (a semi-permeable/partially permeable physical barrier) that is positioned on the top of a cell monolayer on the luminal side LS within the reservoir R to prevent mucus dilution and assist in the formation of a dense layer of mucus. A separator can be impermeable to mucin but permeable to, for example, water. Examples of these separators include, but are not limited to, porous membrane, hydrogels (e.g. agarose, gelatin, collagen, Matrigel®, etc.), porous materials, semi-liquid masses, oils (e.g. mineral oils, perfluorocarbons, etc.), solid floaters (e.g. waxes, plastics, etc.), and meshes (nylon, photoresists, polydimethylsiloxane and other synthetic polymers, etc.). A separator can act as a diffusion barrier to the aqueous media/medium or to the mucus constituents.

Alternatively, in some embodiments, an impermeable physical barrier (a blocker) can also be used, as shown in FIG. 1E, to facilitate the accumulation of a dense mucus layer. The impermeable physical barrier can be positioned on top of (above), i.e. on the luminal side LS, the mucus producing cell monolayer within the reservoir R. The impermeable physical barrier or blocker can be impermeable to mucins and to water so that it confines the mucus accumulation. Examples of blockers include, but are not limited to, hydrogels (e.g. agarose, gelatin, collagen, Matrigel®, etc.), porous materials, oils (e.g. mineral oils, perfluorocarbons, etc.), solid floaters (e.g. waxes, plastics, etc.), and meshes (nylon, photoresists, polydimethylsiloxane and other synthetic polymers, etc.).

Additional methods for generating dense mucus layers on the surface of mucus producing cell monolayers can be used to further enhance the density of the mucus layer. For example, in some embodiments, surface forces or mechanical stimulation can be applied to the surface of the cell monolayer comprising the mucus producing cells to mimic the impact of surface tension forces (without diluting the mucus) that can be present during meniscus formation generated by ALI. Mechanisms for exerting shear forces in a non-dilutive manner can include, but are not limited to, stir bars, mechanical rotation of a layer or material above the cells, back and forth movements parallel to the surface induced by a viscous layer overlaid on the cell surface (this can include providing a rocking motion such as with a mechanical rocker). Mechanical stimulation also can be applied to the cell surface, e.g. within reservoir R on the luminal side LS, to facilitate mucus production and accumulation. Mechanical stimulation can include, but is not limited to, shear stress applied on the apical surface by fluid flow, mechanical scrape of the apical mucus layer, and/or cyclic deformation of cells to mimic peristalsis, etc.

Any combination of the methods described herein can be used to facilitate the formation of a mucus layer above a cell monolayer comprising mucus producing cells. By adjusting the methods and combinations of methods as described herein mucus layers can be generated having different properties including different thicknesses, different densities and regions of different densities/thicknesses.

Accordingly, in some embodiments, the presently disclosed subject matter can provide methods of producing live cell constructs comprising a cell monolayer comprising mucus producing cells and a mucus layer, the method comprising: (a) culturing stem cells that are capable of differentiating into mucus producing cells (e.g., intestinal epithelial stem cells, basal stem cells, induced pluripotent stem cells and the like) on an upper surface of a cell support structure having both an upper surface and a lower surface until at least a portion of the upper surface of the cell support structure is covered by the stem cells; and (b) culturing the stem cells further to produce a cell monolayer comprising mucus producing cells (e.g., goblet cells), the cell monolayer having a basal side and a luminal (apical) side, wherein the mucus producing cells of the cell monolayer establish a mucus layer on the luminal side of the cell monolayer, and wherein the mucus layer is substantially impenetrable to micro-objects, thereby producing a live cell construct comprising a cell monolayer comprising mucus producing cells and a mucus layer. In some embodiments, the mucus layer can have a thickness of about 1 micron to about 1 cm.

In some embodiments, the presently disclosed subject matter can provides methods of producing live cell constructs comprising a cell monolayer comprising mucus producing cells and a mucus layer, the method comprising: (a) culturing stem cells that are capable of differentiating into mucus producing cells (e.g., intestinal epithelial stem cells, basal stem cells, induced pluripotent stem cells and the like) on an upper surface of a cell support structure having both an upper surface and a lower surface until at least a portion of the upper surface of the cell support structure is covered by the stem cells; and (b) culturing the stem cells further to produce a cell monolayer comprising mucus producing cells (e.g., goblet cells), the cell monolayer having a basal side and a luminal (apical) side, wherein the mucus producing cells of the cell monolayer establish a mucus layer on the luminal side of the cell monolayer, the mucus layer having a thickness of about 1 microns to 1 cm, thereby producing a live cell construct comprising a cell monolayer comprising mucus producing cells and a mucus layer. In some embodiments, the mucus layer can be impenetrable or substantially impenetrable to micro-objects.

In some embodiments, the presently disclosed subject matter can provide methods of producing live cell constructs comprising a cell monolayer comprising mucus producing cells and a mucus layer, the method comprising: (a) culturing stem cells that are capable of differentiating into mucus producing cells (e.g., intestinal epithelial stem cells, basal stem cells, induced pluripotent stem cells and the like) on an upper surface of a cell support structure having both an upper surface and a lower surface until at least a portion of the upper surface of the cell support structure is covered by the stem cells; and (b) culturing the stem cells further to produce a cell monolayer comprising mucus producing cells (e.g., goblet cells), the cell monolayer having a basal side and a luminal (apical) side, wherein the mucus producing cells of the cell monolayer establish a first mucus layer and a second mucus layer on the luminal side of the cell monolayer, wherein the first mucus layer is adjacent to and above the second mucus layer and the second mucus layer is adjacent to and above the cell monolayer and the second mucus layer is impenetrable or substantially impenetrable to micro-objects, thereby producing a live cell construct comprising a cell monolayer comprising mucus producing cells and a mucus layer. In some embodiments, the thickness of the second mucus layer can be about 1 micron to about 1 cm, as described further herein.

In some embodiments, a method of producing a live cell construct comprising a cell monolayer comprising mucus producing cells and a mucus layer is provided, the method comprising: (a) culturing stem cells that are capable of differentiating into mucus producing cells (e.g., intestinal epithelial stem cells, basal stem cells, induced pluripotent stem cells and the like) on an upper surface of a cell support structure having both an upper surface and a lower surface until at least a portion of the upper surface of the cell support structure is covered by the stem cells; and (b) culturing the stem cells further to produce a cell monolayer comprising mucus producing cells (e.g., goblet cells), the cell monolayer having a basal side and a luminal (apical) side, wherein the mucus producing cells of the cell monolayer establish a first mucus layer and a second mucus layer on the luminal side of the cell monolayer, wherein the first mucus layer is adjacent to and above the second mucus layer and the second mucus layer is adjacent to and above the cell monolayer and the second mucus layer comprises a thickness of about 1 microns to 1 cm, thereby producing a live cell construct comprising a cell monolayer comprising mucus producing cells and a mucus layer. In some embodiments, the second mucus layer can be impenetrable or substantially impenetrable to micro-objects.

In some embodiments, in the methods of the presently disclosed subject matter a basal reservoir can be defined below the basal side of the cell layer of mucus producing cells and a luminal reservoir can be defined above the luminal side of the cell monolayer comprising mucus producing cells, and the basal reservoir and the luminal reservoir can each comprise a liquid medium; the method further comprising: (a) removing the liquid medium in the luminal reservoir to produce an air-liquid interface at the luminal side of the cell layer that comprises mucus producing cells; and/or (b) adjusting the volume of the liquid medium in the luminal (apical) reservoir to a depth in a range of about 0.001 mm to about 10 mm, optionally about 0.001 mm to about 1 mm, above the luminal side of the cell monolayer. In some embodiments, the mucus layer is present or develops between the liquid medium and the cell monolayer. Thus, in some embodiments, the volume of liquid can be adjusted prior to, during, or after the production of mucus by the mucus producing cells. Once mucus producing cells are present in the live cell construct, at least a thin mucus film can be constitutively produced by the mucus producing cells (e.g., goblet cells). A thick layer of mucus can then accumulate after the adjustment of the volume of liquid in the luminal reservoir. In some embodiments, the mucus layer can be continuous or it can be discontinuous across the surface of the cell monolayer.

In some embodiments, stem cells useful with the present disclosure can include, but are not limited to, epithelial stem cells, intestinal epithelial stem cells, basal stem cells, induced pluripotent stem cells, respiratory stem cells, gastric stem cells, nasal stem cells, reproductive tract cells (cervix, vagina, uterus), urethra cells, olfactory cells, mouth cells, tongue cells, and/or conjunctiva cells. In some embodiments, the stem cells are intestinal epithelial stem cells.

In some embodiments, a live cell construct comprising a cell monolayer comprising mucus producing cells can comprise one or more different cell types (e.g., 1, 2, 3, 4, 5, or more) in addition to mucus producing cells (e.g., goblet cells). A “cell type” as used herein refers to morphologically or phenotypically distinct cell forms within a species. In some embodiments, the cells positioned on a cell support structure can be from healthy, inflamed, or diseased human or animal tissue. In some embodiments, cells useful for making a live cell construct of the presently disclosed subject matter can be from human or animal tissue having a disease comprising mucus misregulation, including but not limited to, inflammatory bowel disease, constipation, cystic fibrosis irritable bowel syndrome, leaky gut syndrome, bacterial overgrowth syndromes, celiac disease, lactose intolerance, excessive gas syndromes, diarrheal diseases, and/or polyps appendicitis.

In some embodiments, a cell layer of the presently disclosed subject matter can be flat, 2-dimenional as illustrated, for example, in FIGS. 1A through 1E. In some embodiments, a cell monolayer of the presently disclosed subject matter can also be folded in a 3-dimentional shape or structure to mimic, for example, the crypt structure or crypt-villus structure of in vivo intestines.

In some embodiments, the methods of the present presently disclosed subject matter can further comprise positioning an impermeable physical barrier and/or a partially permeable (i.e., semi-permeable) physical barrier on or over the luminal side of the cell monolayer comprising mucus producing cells (and on or over/above the mucus layer). In some embodiments, water transit can be regulated by controlling liquid/water movement or water vapor movement.

In some embodiments, the impermeable and/or partially permeable physical barrier can be positioned directly on the luminal side of the cell monolayer comprising mucus producing cells or directly on the mucus layer (if already present). That is, the impermeable and/or partially impermeable physical barrier can be positioned to be in direct contact with the mucus producing cells and/or the mucus layer. Alternatively, liquid medium can be present between the physical barrier and the mucus producing cells and/or the mucus layer when the physical barrier is positioned. In some embodiments, when present between the physical barrier and the mucus producing cells and/or mucus layer, the liquid medium can be present at a depth in a range of about 0.001 mm to about 10 mm, optionally about 0.001 mm to about 1 mm, above the luminal side of the cell monolayer (and/or above the luminal side of the mucus layer) and below the physical barrier.

In some embodiments, the volume of the liquid medium in the luminal (apical) reservoir (with or without a physical barrier), or on the luminal side of the cells, can be a depth in a range of about 0.001 mm to about 10 mm above the luminal side of the cell monolayer (e.g., about 0.001, 0.002, 0.003, 0.004, 0.005, 0.006, 0.007, 0.008, 0.009, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9, 3, 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 10 mm, or any value or range therein) (e.g., about 0.005 mm to about 10 mm, 0.01 mm to about 10 mm, about 0.05 mm to about 10 mm, 0.1 mm to about 10 mm, about 0.5 mm to about 10 mm, 1 mm to about 10 mm, about 5 mm to about 10 mm, about 0.001 mm to about 1 mm, about 0.005 to about 1 mm, 0.01 mm to about 1 mm, about 0.05 mm to about 1 mm, 0.1 mm to about 1 mm, about 0.5 mm to about 1 mm, about 0.001 mm to about 0.1 mm, about 0.005 to about 0.1 mm, 0.01 mm to about 0.1 mm, about 0.05 mm to about 0.1 mm, 0.1 mm to about 0.1 mm, about 0.5 mm to about 0.1 mm, or any value or range therein).

As used herein, a “partially permeable physical barrier” is impermeable or substantially impermeable to mucin, but water can pass through the barrier. Thus, in some embodiments, a partially permeable physical barrier can have a molecular weight cut-off (MWCO) of about 100 kDa, i.e., the barrier is impermeable to molecules greater than (>) about 100 kDa. In some embodiments, the partially permeable barrier can have a MWCO of about 100 to about 150 kDa). Mucin has molecular weight of about 200 kDa-200 MDa.

As used herein an “impermeable physical barrier” is at least substantially, and preferably completely, impermeable to the liquid medium (e.g., water) and to mucin.

Thus, in some embodiments, an impermeable physical barrier or a partially permeable physical barrier can be used to confine mucins on or near the surface of a mucin producing cell monolayer. In some embodiments, an impermeable physical barrier and/or a partially permeable physical barrier can be used to prevent or reduce the dilution by the liquid medium of the mucin as it is produced by the cell monolayer.

Non-limiting examples of physical barriers include a semi-liquid mass (e.g. hydrogels), a gas-impermeable membrane, a gas permeable membrane, and hygroscopic materials (honey, glycerin, sugar, nylon, ABS (acrylonitrile/butadiene/styrene), polycarbonate, cellulose, and poly (methyl methacrylate)). In some embodiments, partially permeable (e.g., molecular weight cutoff of about 100 kDa) physical barriers can include but are not limited to porous materials including porous membranes, some synthetic polymers, hydrogels (e.g., agarose, gelatin, collagen, Matrigel®, etc.), some oils, and/or meshes (e.g., nylon, photoresists, polydimethylsiloxane and other synthetic polymers, etc.). In some embodiments, a vapor permeable (mucus impermeable) physical barrier can be used. Nonlimiting examples of vapor permeable membranes useful with the presently disclosed subject matter include polydimethylsiloxane (PDMS) without coatings/fillers, some synthetic polymers, and/or meshes. Non-limiting examples of impermeable membranes (impermeable to water and to mucin) include solid floaters (e.g., waxes, plastics, etc.), meshes (nylon, photoresists, polydimethylsiloxane and other synthetic polymers, etc.), oils (e.g., mineral oils, perfluorocarbons, natural oils etc.), and/or synthetic polymers.

In some embodiments, the thickness of a mucus layer of a live cell construct of the presently disclosed subject matter can be about 1 micron to about 1 cm (10,000 microns) (e.g., about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 335, 940, 945, 950, 955, 960, 965, 970, 975, 980, 985, 990, 995, 1000, 1100, 1200, 1300, 1400, 1500, 1600, 1700, 1800, 1900, 2000, 2100, 2200, 2300, 2400, 2500, 3000, 3500, 4000, 4500, 5000, 5500, 6000, 6500, 7000, 7500, 8000, 8500, 9000, 9500, 10,000 microns, or any value or range therein). Thus, in some embodiments, the thickness of the mucus layer can be about 2 microns to about 1 cm, about 3 microns to about 1 cm, about 4 microns to about 1 cm, about 5 microns to about 1 cm, about 6 microns to about 1 cm, about 7 microns to about 1 cm, about 8 microns to about 1 cm, about 9 microns to about 1 cm, about 10 microns to about 1 cm, about 20 microns to about 1 cm, about 30 microns to about 1 cm, about 40 microns to about 1 cm, about 50 microns to about 1 cm, about 60 microns to about 1 cm, about 70 microns to about 1 cm, about 80 microns to about 1 cm, about 90 microns to about 1 cm, about 100 microns to about 1 cm, about 200 microns to about 1 cm, about 300 microns to about 1 cm, about 400 microns to about 1 cm, about 500 microns to about 1 cm, about 1000 microns to about 1 cm, about 1500 microns to about 1 cm, about 30 microns to about 7500 microns, about 40 microns to about 7500 microns, about 50 microns to about 7500 microns, about 60 microns to about 7500 microns, about 70 microns to about 7500 microns, about 80 microns to about 7500 microns, about 90 microns to about 7500 microns, about 100 microns to about 7500 microns, about 500 microns to about 7500 microns, about 1000 microns to about 7500 microns, about 30 microns to about 5000 microns, about 40 microns to about 5000 microns, about 50 microns to about 5000 microns, about 60 microns to about 5000 microns, about 70 microns to about 5000 microns, about 80 microns to about 5000 microns, about 90 microns to about 5000 microns, about 100 microns to about 5000 microns, about 500 microns to about 5000 microns, about 1000 microns to about 5000 microns, about 30 microns to about 2500 microns, about 40 microns to about 2500 microns, about 50 microns to about 2500 microns, about 60 microns to about 2500 microns, about 70 microns to about 2500 microns, about 80 microns to about 2500 microns, about 90 microns to about 2500 microns, about 100 microns to about 2500 microns, about 200 microns to about 2500 microns, about 300 microns to about 2500 microns, about 500 microns to about 2500 microns, about 10 microns to about 1000 microns, about 20 microns to about 1000 microns, about 30 microns to about 1000 microns, about 40 microns to about 1000 microns, about 50 microns to about 1000 microns, about 60 microns to about 1000 microns, about 70 microns to about 1000 microns, about 80 microns to about 1000 microns, about 90 microns to about 1000 microns, about 100 microns to about 1000 microns, about 200 microns to about 1000 microns, about 300 microns to about 1000 microns, about 500 microns to about 1000 microns, about 30 microns to about 500 microns, about 40 microns to about 500 microns, about 50 microns to about 500 microns, about 60 microns to about 500 microns, about 70 microns to about 500 microns, about 80 microns to about 500 microns, about 90 microns to about 500 microns, about 100 microns to about 500 microns, about 200 microns to about 500 microns, about 30 microns to about 400 microns, about 50 microns to about 400 microns, about 70 microns to about 400 microns, about 100 microns to about 400 microns, about 50 microns to about 350 microns, about 70 microns to about 350 microns, about 100 microns to about 350 microns, about 50 microns to about 300 microns, about 70 microns to about 300 microns, about 100 microns to about 300 microns, about 30 microns to about 250 microns, about 50 microns to about 250 microns, about 30 microns to about 200 microns, about 50 microns to about 200 microns, about 100 microns to about 200 microns, about 30 microns to about 150 microns, about 50 microns to about 150 microns, about 1 microns to about 100 microns, about 5 microns to about 100 microns, about 10 microns to about 100 microns, about 20 microns to about 100 microns about 30 microns to about 100 microns, about 50 microns to about 100 microns, or any range or value therein.

In some embodiments, a mucus layer produced by mucus producing cells of a live cell construct of the presently disclosed subject matter can be impenetrable or substantially impenetrable to (a population of) micro-objects (e.g., microorganisms or micro-particles (e.g., beads) in a size range from about 0.1 micron to about 100 microns (e.g., about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 microns (μm)) (e.g., about 0.1 to about 5 μm, about 0.1 to about 10 μm, about 0.1 to about 2 μm, about 0.1 to about 30 μm, about 0.1 to about 40 μm, about 0.1 to about 50 μm, about 0.1 to about 60 μm, about 0.1 to about 70 μm, about 0.5 to about 5 μm, about 0.5 to about 10 μm, about 0.5 to about 2 μm, about 0.5 to about 30 μm, about 0.5 to about 40 μm, about 0.5 to about 50 μm, about 0.5 to about 60 μm, about 0.5 to about 70 μm, about 0.5 to about 80 μm, about 0.5 to about 90 μm, about 0.5 to about 100 μm, about 1 to about 5 μm, about 1 to about 10 μm, about 1 to about 2 μm, about 1 to about 30 μm, about 1 to about 40 μm, about 1 to about 50 μm, about 1 to about 60 μm, about 1 to about 70 μm, about 1 to about 80 μm, about 1 to about 90 μm, about 1 to about 100 μm, about 2 to about 5 μm, about 2 to about 10 μm, about 2 to about 20 μm, about 2 to about 50 μm, about 2 to about 70 μm, about 2 to about 80 μm, about 2 to about 90 μm, about 2 to about 100 μm, about 5 to about 20 μm, about 5 to about 50 μm, about 5 to about 70 μm, about 5 to about 80 μm, about 5 to about 90 μm, about 5 to about 100 μm, about 10 to about 20 μm, about 10 to about 50 μm, about 10 to about 70 μm, about 10 to about 80 μm, about 10 to about 90 μm, about 10 to about 100 μm or any amount or range therein). A microorganism useful for measuring penetrability of mucus can be, for example, a bacterium. Non-limiting examples of bacteria that can be used to ascertain the permeability of a mucus layer as is known in the art and can include, for example, Firmicutes, Bacteroidetes, Actinobacteria, Proteobacteria, Bacteroides, Clostridium, Faecalibacterium, Eubacterium, Ruminococcus, Peptococcus, Peptostreptococcus, and Bifidobacterium. In some embodiments, the bacterium can be in the genus Escherichia spp. or Lactobacillus spp.). addition, a useful In micro-objective for determining penetrability/impenetrabiltiy of a mucus layer can be a micro-particle (e.g., bead) having a size of about 0.1 micron to about 100 microns. In general, micro-particles useful for measuring permeability are round and are comprised of one or more polymers (e.g., beads).

As used herein, “substantially impenetrable” means more than about 70% of a population of micro-objects are unable to penetrate the mucus layer (e.g., more than about 70, 71, 72, 73, 74, 75, 76, 77, 78, 79, 80, 81, 82, 83, 84, 85, 86, 87, 88, 89, 90, 91, 92, 93, 94, 95, 96, 97, 98, 99%, and any range or value therein; e.g., about 70% to about 99%, about 75% to about 99%, about 80% to about 99%, about 85% to about 99%, about 90% to about 99%, about 95% to about 99%, and any range or value therein, of the population of micro-objects are unable to penetrate the mucus layer) and/or the micro-objects that penetrate can travel a distance of less than about 30% into the thickness of the mucus layer (or less than 30% of the distance from the luminal side of the mucus layer to the basal side of the mucus layer) (e.g., about 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30%; e.g., about 0.5% to about 30%, about 1% to about 30%, about 5% to about 30%, about 10% to about 30%, about 15% to about 30%, about 20% to about 30%, about 0.5% to about 20%, about 1% to about 20%, about 5% to about 20%, about 10% to about 20%, about 15% to about 20%, about 20% to about 25%, about 0.5% to about 15%, about 1% to about 15%, about 5% to about 15%, about 10% to about 15%, about 0.5% to about 10%, about 1% to about 10%, about 5% to about 10%, about 0.5% to about 5%, about 1% to about 5%, about 2.5% to about 5%, about 0.5% to about 1%, and any range or value therein). In some embodiments, a mucus layer that is substantially impenetrable can be impenetrable to more than 90% (e.g., about 90, 91, 92, 93, 94, 95, 96, 97, 98, 99%, and any range or value therein; e.g., about 90% to about 99%) of a population of micro-objects. In some embodiments, in a mucus layer that is substantially impenetrable can be impenetrable to more than 90%, the micro-objects that penetrate travel less than 10% (e.g., less than 0.5, 0.6, 0.7, 0.8, 0.9, 1, 2, 3, 4, 5, 6, 7, 8, 9, 10%) of the distance from the luminal side of the mucus layer to the basal side of the mucus layer.

As used herein “impenetrable” means more than about 99% (e.g., about 99, 99.1, 99.2, 99.3, 99.4, 99.5, 99.6, 99.7, 99.8, 99.9, 100%, and any range or value therein; e.g., 99-100%) of a population of micro-objects are unable to penetrate the mucus layer and/or the micro-objects that penetrate travel a distance of less than about 10% into the thickness of the mucus layer (e.g., less than 10% of the distance from the luminal side of the mucus layer to the basal side of the mucus layer) (e.g., less than 0.5, 0.6, 0.7, 0.8, 0.9, 1, 2, 3, 4, 5, 6, 7, 8, 9, 10%).

As used herein, a “cell support structure” can be any structure upon which the one or more cells and/or tissue can be positioned and can be organic, inorganic, or a composite thereof including, for example, any porous or mesh membrane.

In some embodiments, a cell support structure can comprise an organic polymer such as collagen, typically in combination with other ingredients as discussed below. In some embodiments, the supports are porous. A support can be provided or mounted on a porous carrier (e.g., a porous membrane, a mesh, an inorganic grid, a hydrogel, or a combination thereof) to lend structural support thereto, as also discussed below. A support can be in any suitable shape or configuration, including flat, tubular, curved, spherical, ellipsoid, etc., including composites there (e.g., to emulate macroanatomical structures).

Thus, a cell support structure useful with the presently disclosed subject matter can include, but is not limited to, a membrane, ECM (extracellular matrix), hydrogel, natural or synthetic polymers, and/or a two-or three-dimensional scaffold and/or any combination thereof. In some embodiments, for example, the bottom wall of a luminal reservoir can be a cell support structure (e.g., a membrane). In some embodiments, a cell support structure can comprise microstructures (e.g., features having a size of less than about 1 mm (e.g., about 100, 200 or 300 microns deep, up to 800 or 1000 microns deep or more, and/or from about 10 or 50 microns wide, up to 100 or 200 microns wide or more; e.g., a microwell, a post, and/or a groove). In some embodiments, a cell support structure can be comprised of, for example, polytetrafluoroethylene (PTFE), polyethylene terephthalate (PET), polycarbonate (PC), polyvinylidiene fluoride (PVDF), polyethersulfone (PES), cellulose acetate, regenerated cellulose, cellulose nitride, nylon, carbon grid, graphene films, glass, Bioglass (e.g., 45S5 Bioglass), hydroxyapatite, calcium phosphate, silicon, silicon oxide, silicon nitride, titanium oxide, aluminum oxide, gold, nickel, and/or stainless steel, or any combination thereof.

In some embodiments, a material useful as a cell support structure of the presently disclosed subject matter that is not naturally porous, can be made porous by methods that include, but are not limited to, sintering, etching, leaching, lithography, laser micromachining, etc. For example, a porous mesh of silicon and gold can be fabricated by lithography/etching. In some embodiments, photoreactive polymers such as photoresist that are fabricated into a film with micro or nanopores or micro or nanomesh by photolithography can be used for a cell support structure. In some embodiments, elastomeric films such as polydimethylsiloxane (PDMS) or EcoFlex that are fabricated into porous film or micro/nanomesh by soft lithography or molding can also be used as cell support structure. In some embodiments, a cell support structure can also be a dehydrated or flexible yet strong matrix such as a collagen or fibrin film or a composite.

Cells and/or tissues can be placed on a cell support structure or scaffold with or without additional adhesion proteins or extracellular matrices. In some embodiments, a scaffold can comprise extracellular matrix (ECM) materials including, but not limited to, collagen, gelatin, laminin, elastin, fibronectin, vitronectin, heparin sulfate, chondroitin sulfate, keratin sulfate, hyaluronic acid, gelatinous protein mixture secreted by Engelbreth-Holm-Swarm mouse sarcoma cells (e.g. Matrigel®, Geltrex®, MaxGel™, etc.), and/or commercially available cell substrates (e.g., CELLstart™ CTS™) and any combination thereof (e.g., a collagen/Matrigel® mixture). In some embodiments, hydrogel from natural polymers, synthetic polymers and hybrid hydrogel can be used to build a scaffold in two dimensions or three dimensions. Examples of natural polymers and synthetic polymers include, but are not limited to, chitosan, agarose, alginate (e.g., AlgiMatrix®), fibrin, silk, polyvinyl alcohol, sodium polyacrylate, acrylate polymers, polyethylene glycol (PEG), synthetic peptides, poly N-isopropylacrylamide, and/or polyacrylamide, and/or any combination thereof. In some embodiments, the surface of a scaffold can be engineered to promote cell adhesion with any one or a combination of ECM molecules, natural or synthetic polymers or synthetic peptides including, but not limited to, poly-l-lysine, RGD-peptide and other integrin recognizing peptide segments. In some embodiments, a cell support structure useful with this presently disclosed subject matter can be mixed with cellular materials (immune cells or other cell types, tissues, blood), or non-cellular materials (drugs, polymer beads, magnetic particles, etc.). In some embodiments, a cell support structure can comprise a two or three dimensional micropatterns or microstructures.

In some embodiments, the cells of the live cell construct can be cultured in liquid medium comprising, for example, an additive, a compound, and/or a solution that can contribute to water balance across the cell layer, thereby assisting in formation of the mucus layer. In some embodiments, the additive, compound, and/or solution can include, but is not limited to, a hormone, a chemical additive, a food additive, a bacterial metabolite, and/or a hypertonic salt solution. In some embodiments, the additive, compound, and/or solution can be present in/introduced into a luminal reservoir (luminal side of the cell monolayer) and/or into a basal reservoir (basal side of the cell monolayer). Thus, for example, a hormone that stimulates secretion of water and electrolytes to the intestinal lumen can be added to the basal side of the cell monolayer (basal reservoir) to assist in the balance of fluid movement across the cell monolayer. In contrast, food additives and bacterial metabolites can be added to the luminal side of the cell monolayer (luminal reservoir).

In some embodiments, a hormone useful with this presently disclosed subject matter can include, but is not limited to, a vasoactive intestinal peptide (VIP), 5-hydroxytryptamine (serotonin, 5-HT), substance P, bone morphogenetic protein (BMP), gastrin, cholecystokinin, secretin, ghrelin, motilin, gastric inhibitory polypeptide, leptin, glucagon-like peptides, somatostatin, and/or neurotensin.

Non-limiting examples of useful chemical additives include, but are not limited to, butyrate, dibenzazepine, gamma secretase inhibitor (DAPT, LY411575), forskolin, guaifenesin, carbachol, prostaglandins, phorbal ester (phorbol 12-myristate 13-acetate), histamine, and/or N-(1-oxobutyl)-cyclic 3′, 5′-(hydrogen phosphate) 2′-butanoate-adenosine, monosodium salt (i.e.,dibutyryl-cAMP, sodium salt) (CAS 16980-89-5).

Exemplary food additives include N-nitrosoanabasine, matairesimol and/or caffeine.

In some embodiments, a bacterial metabolite can include, but is not limited to, a short chain fatty acid.

In some embodiments, a salt in a hypertonic salt solution that is useful with the presently disclosed subject matter can include, but is not limited to, to sodium, chlorine, potassium, magnesium, phosphate, carbonate, and/or lithium. In some embodiments, the concentration of the salt in the hypertonic solution can be about 1 mM to about 1000 mM (e.g., about 1, 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 400, 405, 410, 415, 420, 425, 430, 435, 440, 445, 450, 455, 460, 465, 470, 475, 480, 485, 490, 495, 500, 505, 510, 515, 520, 525, 530, 535, 540, 545, 550, 555, 560, 565, 570, 575, 580, 585, 590, 595, 600, 605, 610, 615, 620, 625, 630, 635, 640, 645, 650, 655, 660, 665, 670, 675, 680, 685, 690, 695, 700, 705, 710, 715, 720, 725, 730, 735, 740, 745, 750, 755, 760, 765, 770, 775, 780, 785, 790, 795, 800, 805, 810, 815, 820, 825, 830, 835, 840, 845, 850, 855, 860, 865, 870, 875, 880, 885, 890, 895, 900, 905, 910, 915, 920, 925, 930, 335, 940, 945, 950, 955, 960, 965, 970, 975, 980, 985, 990, 995, 1000 mM, and any value or range therein). Thus, in some embodiments, the concentration of the salt in the solution can be about 5 mM to about 50 mM, about 5 mM to about 100 mM, about 10 mM to about 100 mM, about 10 mM to about 250 mM, about 10 mM to about 500 mM, about 10 mM to about 1000 mM, about 50 mM to about 100 mM, about 50 mM to about 500 mM, about 50 mM to about 1000 mM, about 100 mM to about 250 mM, about 100 mM to about 500 mM, about 100 mM to about 1000 mM, about 200 mM to about 500 mM, about 200 mM to about 1000 mM, about 300 mM to about 500 mM, about 300 mM to about 800 mM, about 300 mM to about 1000 mM, about 400 mM to about 500 mM, about 400 mM to about 800 mM, about 400 mM to about 1000 mM, about 500 mM to about 750 mM, about 500 mM to about 1000 mM, about 600 mM to about 1000 mM, about 700 mM to about 1000 mM, about 800 mM to about 1000 mM, and any value or range therein.

Any substance useful for the growth/maintenance of a cell and/or tissue can be introduced into a basal reservoir or luminal reservoir. In some embodiments, a substance can include, but is not limited to, fibronectin; laminin; epidermal growth factor (EGF); R-spondin; noggin; cytokines (e.g., interleukin (e.g., IL-6, IL-17, IL-22), tumor necrosis factor (TNF)); ephrin receptors (e.g., EphrinB, EphBs); bone morphogenetic proteins (BMPs. BMP-2, BMP-7); Wnt (wingless-related integration site) (e.g., Wnt3, Wnt3A, and other Wnts); notch signaling factors (notch receptors); DI11/4; Noggin; Grem1; Grem2; acetate; butyrate; proprionate, desaminotyrosine, catecholamine (e.g., dopamine, norepinephrine) cytokines, and/or short chain fatty acids.

In some embodiments, to assist in establishing a mucus layer, a force can be applied, e.g., parallel to the surface of the cell layer. In some embodiments, the force can be, for example, application of a surface tension force (e.g., air liquid interface (ALI)) or it can be by application of a mechanical force. Non-limiting examples of application of a mechanical force include movement generated by a stir bar, moving a semi-solid material (e.g., hydrogel) parallel to the cell surface, and/or circulating a slurry on the top of the cell surface.

The presently disclosed subject matter further provides a live cell construct comprising a cell monolayer comprising mucus producing cells and a mucus layer, wherein the mucus layer is impenetrable or substantially impenetrable to micro-objects (e.g., microorganisms (e.g., bacteria) or micro-particles (e.g., beads)). In some embodiments, the mucus layer can be impenetrable or substantially impenetrable to micro-objects in a size range from about 0.1 to about 100 microns. In some embodiments, the mucus layer can be about 1 micron to about 1 cm in thickness (depth). In some embodiments, the mucus layer of a live cell construct of the presently disclosed subject matter can comprise a basal side and a luminal side, wherein the basal side is below the mucus layer and adjacent to (directly above) the mucus producing cells and the luminal side is above the mucus and the mucus producing cells and adjacent to (directly below) liquid medium or the air in the luminal reservoir.

The presently disclosed subject matter further provides methods of using the live cell construct of the presently disclosed subject matter, including but not limited to, studying (a) the ability of an organism, a drug, or a particle to traverse (penetrate) a mucus layer of a cell; (b) an immunological response of a cell comprising a mucus layer to invasion by an organism or contact with a particle and or chemical/compound; (c) the ability of an organism to infect a cell comprising a mucus layer; (d) the effectiveness of a drug to prevent infection by an organism or reduce the ability of an organism to infect; (e) misregulation of mucus in diseases including, but not limited to, inflammatory bowel disease, constipation, cystic fibrosis, irritable bowel syndrome, leaky gut syndrome, bacterial overgrowth syndromes, celiac disease, lactose intolerance, excessive gas syndromes, diarrheal diseases, and/or polyps appendicitis.

Thus, in some embodiments, the presently disclosed subject matter provides a method of determining the ability of an organism, a drug, or a particle to traverse (penetrate) a mucus layer of a cell monolayer, comprising: contacting the luminal side of the mucus layer of the live cell construct of the presently disclosed subject matter with the organism, drug, or particle; and measuring the distance that the organism, drug, or particle moves into/across the mucus layer (e.g., from the luminal side of the mucus layer to the basal side of the mucus layer), thereby determining the ability of the organism, drug or particle to traverse (penetrate) the mucus layer of the cell monolayer of the live cell construct. In some embodiments, the distance that the organism, the drug or the particle moves into the mucus layer can be measured over time, thereby determining the rate of movement of the organism, drug or particle into/across the mucus layer of the cell monolayer of the live cell construct.

In some embodiments, a method of studying and evaluating the ability of an organism to infect a cell monolayer comprising a mucus layer is provided, the method, comprising: contacting the luminal side of the mucus layer of the live cell construct of the presently disclosed subject matter with the organism; and determining if the organism traverses the mucus layer and contacts the cell monolayer of the live cell construct, wherein when the organism is determined to traverse the mucus layer and contact the cell monolayer, the organism is determined to be able to infect the cell monolayer comprising a mucus layer.

In some embodiments, a method of evaluating the effectiveness of a drug to prevent infection by an organism or to reduce the ability of an organism to infect is provided, comprising: contacting the luminal side of the mucus layer of the live cell construct of the presently disclosed subject matter with the organism; contacting the luminal side of the mucus layer of the live cell construct with the drug, and determining whether the organism penetrates the mucus layer and/or infects one or more cells of the cell monolayer of the live cell construct, wherein the drug is determined to be effective in preventing infection or reducing the ability of an organism to infect if the organism does not penetrate the mucus layer and/or does not infect one or more cells of the cell monolayer of the live cell construct as compared to a control (i.e., contacted with the organism but no drug). In some embodiments, wherein contacting the luminal side of the mucus layer of the live cell construct with the organism is prior to, concurrent with, or after contacting the luminal side of the mucus layer of the live cell construct with the drug. In some embodiments, a drug can be determined to be effective when about 25% to about 100% of the organisms are prevented from penetrating the mucus layer and/or are killed (e.g., about 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% are prevented from penetrating the mucus layer and/or are killed, and any range or value therein.

In some embodiments, the presently disclosed subject matter provides a method of evaluating an immunological response of a cell monolayer comprising a mucus layer to invasion by an organism, contact by a particle, and or contact by a chemical/compound, comprising: contacting the luminal side of the mucus layer of the live cell construct of the presently disclosed subject matter with the organism, particle and or chemical/compound; and assaying cells of the cell monolayer of the live cell construct for the production of a marker associated with an immune response (e.g., a cytokine, a chemokine, a hormone, a neurotransmitter, and/or a antimicrobial peptide), thereby evaluating the immunological response of the cell monolayer of the live cell construct to contact by the organism, particle and or chemical/compound.

In some embodiments, a chemical and/or compound can include, but is not limited to, a dietary metabolite and/or a bacteria metabolite such as vitamins or short chain fatty acids.

An organism that can be studied using the methods and live cell constructs of the present presently disclosed subject matter can be any organism and includes, for example, a bacterium, a virus, a fungus, protozoan, and/or a helminth. Thus, for example, any bacterium, virus, fungus, protozoan, or helminth can be studied for its ability to penetrate a cell's mucus layer, to evaluate the effectiveness of a drug to prevent infection by the organism/reduce the ability of an organism to infect, and/or to evaluate an immunological response of the cells of the live cell construct in response to contact by the organism.

In some embodiments, the organism can be a bacterium. Non-limiting examples of bacteria include those from the genus Escherichia spp., Yersinia spp., Salmonella spp., Campylobacter spp., Clostridium spp., Helicobacter spp., Bacteroides spp., Peptostreptococcus spp., Vibrio spp., Shigella spp., Salmonella spp., Listeria spp. and Staphylococcus spp. In some embodiments, a bacterium can include, but is not limited to, Acinetobacter baumannii, Actinomyces israelii, Bacillus anthracis, Bacteroides fragilis, Bartonella henselae, Bordetella pertussis, Borrelia burgdorferi, Borrelia garinii, Borrelia afzelil, Borrelia recurrentis, Brucella abortus, Brucella canis, Brucella melitensis, Brucella suis, Burkholderia pseudomallei, Campylobacter jejuni, Chlamydia pneumoniae, Chlamydia trachomatis, Chlamydophila psittaci, Clostridium botulinum, Clostridium difficile, Clostridium perfringens, Clostridium tetani, Corynebacterium amycolatum, Corynebacterium diphtheriae, Coxiella burnetii, Ehrlichia canis, Ehrlichia chaffeensis, Enterococcus faecalis, Enterococcus faecium, Escherichia coli, Enterotoxigenic Escherichia coli, Enteropathogenic Escherichia coli, Enteroinvasive Escherichia coli, enterohemorrhagic Escherichia coli, Francisella tularensis, Haemophilus influenzae, Helicobacter pylori, Klebsiella pneumoniae, Legionella pneumophila, Leptospira species, Listeria monocytogenes, Mycobacterium leprae, Mycobacterium tuberculosis, Mycoplasma pneumoniae, Neisseria gonorrhoeae, Neisseria meningitidus, Parachlamydia, Pseudomonas aeruginosa, Nocardia asteroides, Rickettsia rickettsii, Salmonella bongori, Salmonella enterica, Staphylococcus aureus, Staphylococcus epidermidis, Staphylococcus saprophyticus, Streptococcus agalactiae, Streptococcus pneumoniae Streptococcus pyogenes, Streptococcus viridans, Treponema pallidum, Vibrio cholerae, Vibrio vulnificus, Vibrio parahaemolyticus, and/or Yersinia pestis.

In some embodiments, the organism can be a protozoan. Non-limiting examples of protozoa include those from the phylum of Amoebozoa, Excavata, and/or Chromalveolata. In some embodiments, a protozoan can include, but is not limited to, those from the genus Amoeba spp., Entamoeba spp., Plasmodium spp., Giardia spp., and/or Trypanosoma spp. In some embodiments, a protozoan can include, but is not limited to, Entamoeba histolytica, Cryptosporidium parvum, Cryptosporidium hominis, Cyclospora cayetanensis, and/or Giardia lamblia

In some embodiments, the organism can be a virus. Non-limiting examples of viruses include Simplexvirus, Varicellovirus, Cytomegalovirus, Roseolovirus, Lymphocryptovirus, Rhadinovirus, Adenovirus, Astrovirus, Calicivirus, Mastadenovirus, Alphapapillomavirus, Betapapillomavirus, Gammapapillomavirus, Mupapillomavirus, Nupapillomavirus, Polyomavirus, Molluscipoxvirus, Orthopoxvirus, Parapoxvirus, Alphatorquevirus, Betatorquevirus, Gammatorquevirus, Gemycircularviruses, Erythrovirus, Dependovirus, Bocavirus, Coltivirus, Rotavirus, Seadornavirus, Hepevirus, Alphacoronavirus, Betacoronavirus, Torovirus, Mamastrovirus, Norovirus, Sapovirus, Flavivirus, Hepacivirus, Pegivirus, Cardiovirus, Cosavirus, Enterovirus, Hepatovirus (e.g., hepatitis A), Kobuvirus, Parechovirus, Rosavirus, Salivirus, Alphavirus, Rubivirus, Deltavirus, Lyssavirus, Vesiculovirus, Filoviridae, Ebolavirus, Marburgvirus, Paramyxoviridae, Henipavirus, Morbilivirus, Respirovirus, Rubulavirus, Metapneumovirus, Pneumovirus, Arenavirus, Peribunyaviridae, Orthobunyavirus, Hantavirus, Nairovirus, Phenuiviridae, Phlebovirus, Influenzavirus A Influenzavirus B, Influenzavirus C, Thogotovirus, Gammaretrovirus Deltaretrovirus, Lentivirus, Spumavirus, and/or Orthohepadnavirus,

In some embodiments, the organism can be a helminth including, but not limited to, intestinal flukes, round worms, pin worms, and/or tape worms. In some embodiments, the helminth can include, but is not limited to, a helminth from the genus of Ascaris spp., Ancylostoma spp., Trichuris spp, Strongyloides spp., Necator spp., Schistosoma spp., and/or Trichinella spp. Further non-limiting examples of helminths include Ascaris lumbricoides (roundworm), Ancylostoma duodenale (hookworm), Necator americanus (hookworm), Strongyloides stercoralis, Trichinella spiralis and/or Trichuris trichiura (whipworm).

In some embodiments, the organism can be a fungus. Non-limiting examples of fungi include those from the genus Candida spp., Aspergillus spp., Mucor spp., Fusarium spp., Blastomyces spp., Coccidioides spp., Cryptococcus spp., Histoplasma spp., Rhizopus spp., Lichtheimia spp., Pneumocystis spp., Sporothrix spp. and/or Cunninghamella spp. Further non-limiting examples of fungi include Candida albicans, Candida tropicalis, Aspergillus flavus, Aspergillus fumigatus, Aspergillus niger, Cryptococcus neoformans, Cryptococcus gattii, Pneumocystis jirovecii, and/or Torulopsis, glabrata.

The presently disclosed subject matter further provides a method of evaluating mucus misregulation in an in vitro cell system. In some embodiments, the method of evaluating mucus misregulation in an in vitro cell system comprises generating a live cell construct of the presently disclosed subject matter from stem cells from subjects having diseases associated with mucus misregulation. In some embodiments, a live cell construct of the present presently disclosed subject matter can be generated from stem cells from a healthy subject that are modified to recapitulate cells from diseases associated with mucus misregulation via genome editing (e.g. CRISPR-Cas9, TALEN, meganuclease). The mucus layers from the in vitro cell systems so generated can then be studied for characteristics including, but not limited to, thickness, composition, viscosity, degree of penetration by micro-objects, ability of microorganisms to infect, and/or responsiveness to drugs as described herein. Diseases associated with mucus misregulation include, but are not limited to, inflammatory bowel disease, constipation, cystic fibrosis irritable bowel syndrome, leaky gut syndrome, bacterial overgrowth syndromes, celiac disease, lactose intolerance, excessive gas syndromes, diarrheal diseases, and/or polyps appendicitis.

The presently disclosed subject matter will now be described with reference to the following examples. It should be appreciated that these examples are not intended to limit the scope of the claims to the presently disclosed subject matter, but are rather intended to be exemplary of certain embodiments. Any variations in the exemplified methods that occur to the skilled artisan are intended to fall within the scope of the presently disclosed subject matter.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-4

In vitro expansion of human colonic epithelial stem cells on a collagen hydrogel. To expand human-derived intestinal epithelial stem cells, a monolayer culture technique was used according to a previously published protocol.^{27, 30} Briefly, colonic crypts were isolated from transverse colon tissue specimens of a cadaveric donor (male, 23 years old), and plated directly on collagen hydrogel at a density of 1,000 crypts/well of a standard 6-well culture plate (possessing a 1 mm-thick collagen hydrogel) and overlaid with 4 mL stem medium (SM-Table 1). The medium was changed every 48 hours. When the cell confluency reached ≥80% (typically 5-7 days), the monolayers were passaged and sub-cultured on fresh collagen hydrogel at a passage ratio of 1:3.^{27, 30} The cells were karyotyped at P11 and 10 out of 10 spreads displayed normal karyotype. All experiments herein used cells at passage numbers less than P15.

Generation of a mucus layer on a monolayer of human colonic epithelium. Transwell inserts possessing a porous membrane (0.4 μm pore size, Corning, #3460) were coated with 1 vol % Matrigel in phosphate-buffered saline (PBS) at 37° C. overnight. The inserts were rinsed with PBS ×1 prior to cell plating. Intestinal epithelial cells were passaged according to the procedure described above except that the cells were suspended in expansion medium (EM-Table 1) and plated directly onto the top compartment of Transwell inserts. Cells from 1 well of the 6-well plate were dispersed into 6 separate 12-well Transwell inserts (1 mL in the upper [apical] reservoir, and 2 mL in the lower [basal] reservoir). The medium was exchanged every 48 h. To induce cell differentiation and mucus production, the medium was switched to differentiation medium (DM-Table 1) after 5 days. In the submerged culture, 1 mL DM was added in the apical reservoir and 2 mL in the basal reservoir. The medium was exchanged every 48 h thereafter. In the ALI culture, the medium in the apical reservoir was completely aspirated, 1 mL DM or DM-VIP (DM containing 330 ng/mL VIP [AnaSpec, #AS-22872]) was added to the basal reservoir, and medium was exchanged every 24 h thereafter. By day 10, the system was suitable for characterization and cytokine assay.

TABLE 1
Formulation of culture media for human colonic epithelial cells

| | SM | | EM | | DM | | DM-VIP | |
|---|---|---|---|---|---|---|---|---|
| WRN-conditioned | 50 | vol % | 50 | vol % | | | | |
| medium | | | | | | | | |
| Advanced DMEM/F12 | 50 | vol % | 50 | vol % | 100 | vol % | 100 | vol % |
| GlutaMax | 1 × | | 1 × | | 1 × | | 1 × | |
| HEPES | 10 | mM | 10 | mM | 10 | mM | 10 | mM |
| Murine EGF | 50 | ng/mL | 50 | ng/mL | 50 | ng/mL | 50 | ng/mL |
| N-acetyl cysteine | 1.25 | mM | 1.25 | mM | 1.25 | mM | 1.25 | mM |
| Primocin | 50 | μg/mL | 50 | μg/mL | 50 | μg/mL | 50 | μg/mL |

| B27 | 1 × | | 1 × | |
|---|---|---|---|---|
| Gastrin | 10 | nM | 10 | nM |

| | | | | | | |
|---|---|---|---|---|---|---|
| A83-01 | 500 | nM | 500 | nM | 500 | nM |

| | | | | |
|---|---|---|---|---|
| SB202190 | 3 | μM | 3 | μM |
| Y-27632 | 10 | μM* | 10 | μM* |

| | | |
|---|---|---|
| Nicotinamide | 10 | mM |
| PGE2 | 10 | nM |

| | | |
|---|---|---|
| VIP | 330 | ng/mL |

*Used in the first 48 hours after cell plating to prevent dissociation-induced cell apoptosis.

Characterization of human colonic epithelium and mucus layer. The cells and mucus layer were fixed with Carnoy's solution (ethanol 6: acetic acid 3: chloroform 1, v/v/v) at 4° C. for 2 h. The tissues were dehydrated with ethanol, and embedded in paraffin for sectioning. The sections were stained with anti-Muc2 antibody (Santa Cruz, #sc-15334) to reveal the mucus layer (Muc2 is the major structural component in colonic mucus layer), and DNA in the nuclei of the cells was stained with Hoechst 33342 (ThermoFisher, #62249).³⁰ To reveal the mucus layer and apical features of the cells, the tissues were fixed by either Carnoy's solution (for submerge and ALI culture) or glyoxal (for ALI culture with DM-VIP) at 4° C. for 2 h, dehydrated in a graded ethanol (25%, 50%, 75% and 100%), dried with a critical point dryer (Tousimis Semidri PVT-3), coated with 10-nm metal by a sputter coater (Cressington 108), and inspected by SEM (FEI Quanta 200 ESEM, FEI Company).

To demonstrate the bacterial separating capability of mucus layer, the live epithelial cells were first stained with Hoechst 33342 by adding 1 mL medium containing 2 μg/mL of Hoechst 33342 to the basal reservoir for 1 h, and then 0.5 mL of a suspension of GFP EC (ATCC, #25922GFP) at a density of 200 million colony-forming units (cfu)/mL, or 1 μm red fluorescent beads (ThermoFisher, #F13083) at a density of 10⁸ beads/mL was added to the apical reservoir. After seeding for 20 min, the Transwell insert was placed on a cover glass and the tissue was imaged with an Olympus FluoView FV3000 confocal laser scanning microscope.

Toxin A experiment. 20 μL of a mixture of natural C. difficile toxin A protein (Abcam, #ab123999) at 0 or 12 μg/mL and FITC-dextran (Sigma, #FD40S) at 5 mg/mL was added to the apical side of the human colonic epithelial monolayers (±mucus) which spread to form a 180 μm liquid layer. 1 mL Hanks' balanced salt solution (with calcium/magnesium, supplemented 10% fetal bovine serum and 10 mM HEPES) was added to the basal compartment. 150 μL samples were collected from the basal compartment at 2, 4, 8 and 24 h. The fluorescence intensity of the collected samples was measured in a microplate reader and the percent permeability was calculated as: % permeability=100×(sample−blank)/(transwell−blank), where “transwell” indicates the liquid collected from inserts without cells. The cells were fixed by ethanol and stained with phalloidin (ThermoFisher, #R37110) and ZO-1 antibody (ProtenTech, #21773-1-AP) to reveal the F-actin and tight junctions.

Co-culture with E. coli and PBMCs. Fresh normal human PBMCs were purchased from Physician's Plasma Alliance (Johnson City, TN). PBMCs were suspended in RPMI medium containing 10% fetal bovine serum (FBS) and 100 μg/ml gentamicin at 2.86 million cells/mL. GFP EC was cultured in a nutrient broth with 100 μg/mL ampicillin. A suspension of GFP EC at a density of 200 million cfu/mL was prepared in 10 mL phosphate-buffered saline (PBS), centrifuged at 2300 g and washed twice with PBS. GFP EC were re-suspended in 0.2 mL RPMI medium containing 10% FBS. Gentamicin (100 μg/ml) was added to the medium to avoid uncontrolled bacterial growth.⁵⁴ For the colonic monolayers under submerged culture in DM for 5 days, the medium was aspirated from both top and bottom reservoirs. For those under ALI culture in DM-VIP for 5 days, the medium was aspirated from the bottom reservoir only. 20 μL GFP EC suspension (20 million cfu) was added to the apical side of the epithelium. 500 μL PBMCs suspension (1.43 million) was added to the basal reservoir. After 24 h of co-culture, the media from the basal reservoirs were collected, centrifuged at 5000 rpm for 6 min, aliquoted and stored at −20° C.

Quantification of cytokines. The concentrations of cytokines (IL-8, IL-6, IL-1β and TNF-α) were determined using ELISA kits (ThermoFisher) according to the manufacturer's instructions. The samples were diluted 40× (for IL-8), 10× (for IL-6) or 5× (for IL-1β and TNF-α) so that the measurements fell within the linear range for a given kit. Three samples were used for each condition. The change of cytokine concentration compared to untreated control was analyzed statistically by two-tailed unpaired t-test. In all figures, ‘**’ denotes p<0.005, ‘*’ p<0.05, and ‘#’ not statistically significant.

### Example 1

### An Air-Liquid Interface (ALI) Culture Enables a Compact Mucus Layer to Accumulate on the Apical Colonic Epithelium

Initially, an air-liquid interface (ALI) culture was tested to aid in generation of a mucus layer as it was hypothesized that the overlying media under standard submerged culture might be diluting the mucus as it was secreted preventing a dense mucus layer from forming. Human epithelial stem cells obtained from the transverse colon were plated on a Matrigel-coated Transwell (porous membrane) and cultured under expansion medium (EM) for 5 days to enable cells to proliferate and form a confluent monolayer. At day 5, the medium was switched to differentiation medium (DM), and the cells were cultured for an additional 5 days as either a submerged culture (medium in both apical and basal reservoirs) or as an ALI culture (medium in basal reservoir only) (FIG. 2A). Due to the absence of growth factors in DM, the cells lost their proliferative capacity and spontaneously differentiated to mature cell lineages composed of a mixture of colonocytes, goblet cells and enteroendocrine cells.²⁹ At day 10 of culture, the mucus layer was absent in the submerged culture (FIG. 2B). As shown in FIG. 2C, when viewed by scanning electron microscopy (SEM), no mucus layer was visible over these monolayers and the epithelium and its apical features (secretory granules of goblet cells, arrows) were readily apparent (FIG. 1C, top panel). As described previously, dissolved mucins were detectable in the luminal medium by enzyme-linked immunosorbent assay (ELISA),³⁰ thus it was possible that the mucins secreted by the goblet cells were rapidly diluted in the medium and thus unable to build a dense, physiologic hydrogel.

In the ALI culture, a continuous mucus layer (Muc2⁺) was observed by immunofluorescence along the entire apical surface of the epithelium (FIG. 2B). The thickness of this mucus layer was heterogeneous and ranged from 76 to 154 μm. The mucus was sometimes associated with goblet cells (indicated with arrows in FIG. 2B). When viewed by SEM, the mucus demonstrated continuous coverage of the epithelium (FIG. 2C, bottom panel) with interspersed fractures due to the dehydration process required for SEM imaging. Cells could not be visualized under the dense mucus layer (high magnification image in the right panel). In addition to the potential for dilution, a variety of mechanisms which impact mucus production can be operational to enhance mucus secretion as seen here. ALI culture has been shown to increase mucus production in the respiratory system and some tissue-cultured tumor cell lines derived from the stomach and intestine,³¹⁻³⁷ although this effect has not been demonstrated previously using primary intestinal tissue. Mechanical forces in combination with ALI have been shown to enhance mucus production by a bed of tumor cells, so surface tension forces exerted by a thin fluid layer can be a contributing factor.³⁷ Evaporation can also provide an additional stimulus due to an increase in the osmolality of the thin residual water film, since hyperosmolar solutions have been shown to cause mucus hypersecretion in human bronchial epithelial cells.^{38, 39} Lastly, changes in osmolality are also known to stimulate production of trefoil factor 3 by colonic epithelial cells, which acts to improve mucus quality and density.^{8, 40}

To determine whether the compact mucus layer served as a barrier to effectively separate microbeads or bacteria from the epithelium, a suspension of 1 μm red fluorescent beads (FIG. 2D) or green fluorescent protein (GFP)-expressing E. coli (GFP EC) (FIG. 2E) was added to the luminal reservoir after ALI culture of an epithelium stained with Hoechst 33342. The thickness of the mucus layer was then evaluated by confocal microscopy (FIGS. 2D and 2E). The distance of the beads and bacteria from the epithelial cells ranged from 71 to 381 μm in the representative images shown in FIGS. 2D and 2E, with an average of 138±62 μm (N=3 locations). While this mucus layer effectively acted as a barrier to separate microbeads or bacteria, it formed mucus clumps with a heterogeneous thickness.

### Example 2

### A Hydrated, Thick Mucus Layer can be Created by Promoting Luminal Water Secretion in a Modified ALI Culture

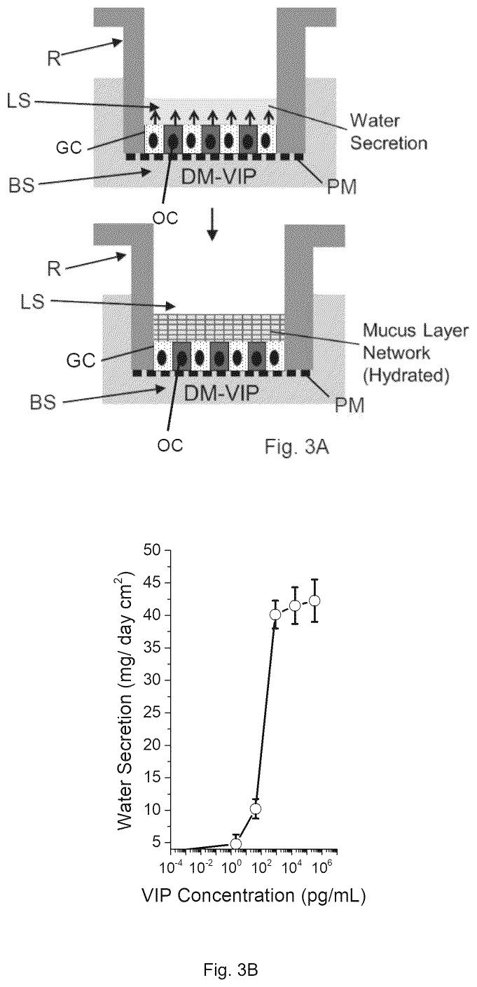

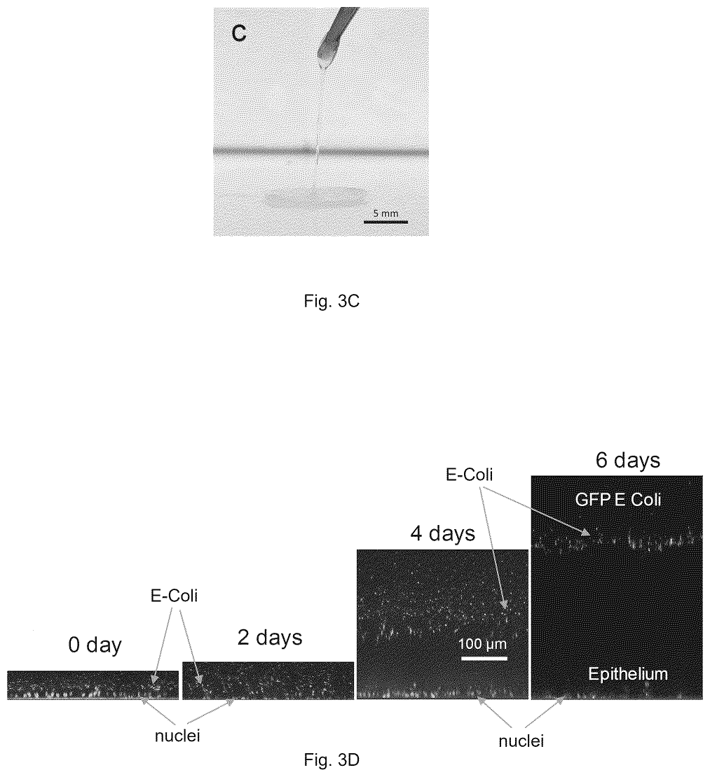

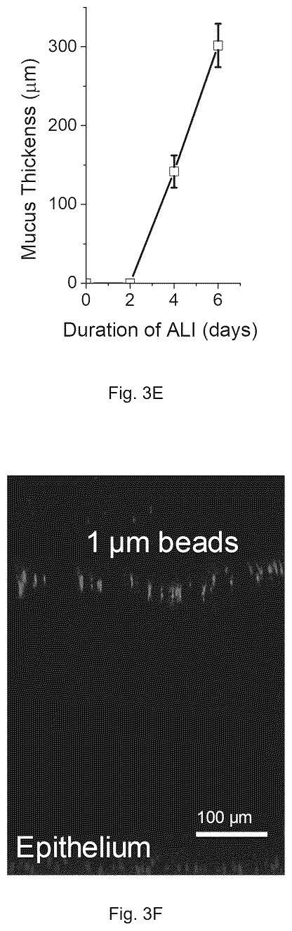

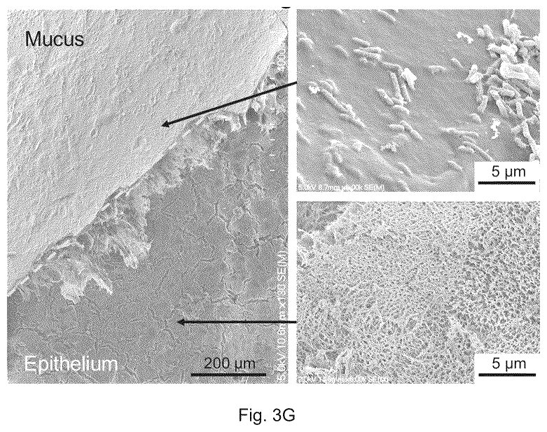

Gas originating from bacterial metabolism is present within the lumen of the large intestine; however, the luminal surface in vivo is predominantly in contact with indigestible materials and waste products possessing a high water content. Thus, in some embodiments the ALI culture described above does not fully reflect the in vivo intestinal luminal environment due to the absence of water at the apical surface. Indeed, water and electrolyte homeostasis of the colonic mucosa are balanced with water moving into and out of the lumen. For healthy adults, the rate of movement of water out of the lumen is 17.8 mL/min and that into the lumen is 16 mL/min, resulting in a net water efflux of 1.85 mL/min causing solidification of the feces.⁴¹ Intestinal hormones, such as 5-hydroxytryptamine (serotonin; 5-HT), vasoactive intestinal peptide (VIP) and substance P play major roles in regulating the fluid balance or water content of the luminal content of the large intestine.⁴² To develop a strategy to produce a thick, highly hydrated, uninterrupted mucus layer of uniform thickness, exposure of the basal epithelial surface to the hormone VIP, using VIP containing DM (DM-VIP), was used to assist in the balance of fluid movement across the epithelium (FIG. 3A). VIP is an endogenous hormone with a plasma concentration in healthy adults ranging from 14 to 76 pg/mL.⁴³ Its role in the intestine is to stimulate secretion of water and electrolytes into the intestinal lumen.⁴⁴ VIP also can act to increase mucus secretion and production by goblet cells as well as enhance lineage allocation towards goblet cells.⁴⁵

To verify the function of VIP to stimulate water secretion, human colonic stem cells were cultured under EM for 5 days. At day 5, the basal medium was switched to DM with varying VIP concentration and the cells placed under the ALI culture conditions. After 24 h, the water accumulated in the apical reservoir was collected and weighed with an analytical balance. In the absence of VIP, the accumulation of water in the apical reservoir was not measurable. In the presence of VIP, the secretion of water was VIP concentration dependent with an effective dose 50 (ED₅₀) of 210 pg/mL (FIG. 3B). A plateau was reached when VIP concentration was >1 ng/mL. At this saturating concentration, approximately 42 mg/cm² water accumulated after a 24 h incubation with VIP, corresponding to a water depth in the reservoir of 420 μm.

Under ALI culture with DM-VIP (DM containing a saturating concentration of VIP), a layer of liquid accumulated on the apical side within 24 h, and this liquid layer was maintained during the 5-6 days of differentiation culture conditions. The apical liquid layer became more and more viscous, and at 5 days a slippery mucus hydrogel was generated which could be lifted off the epithelium by forceps for visualization (FIG. 3C). Under these conditions, the mucus thickness was dependent on the duration of mucus accumulation (i.e., time under ALI plus DM-VIP). To demonstrate the accumulation of the mucus layer over time, human colonic epithelial cells were maintained in a submerged culture in EM for 5 days, followed by culture in DM-VIP for 6 days under the ALI conditions described above. Confocal imaging was performed at 0, 2, 4 and 6 days. Cell nuclei in the monolayer were stained with Hoechst 33342. A suspension of GFP-EC was overlaid onto the mucus layer as described and the distance between the bacteria and epithelial cells measured as described for the micro-beads. The distance between the E. coli (labelled with arrows) and the nuclei (labelled with arrows) show the mucus layer component effectively separated the bacteria from the epithelium (FIG. 3D) over time. At 0 and 2 days, the bacteria were in contact with the apical surface of the epithelium. After 4 days, the bacteria-separating mucus layer was 142±21 μm (N=3 samples) in thickness. After 6 days, the thickness of the mucus layer had increased to 302±28 μm as shown by the separation between the bacteria and the epithelial monolayer. See FIG. 3D. These results demonstrate that the mucus thickness can be readily adjusted by the duration of ALI (FIG. 3E). The thickness of the mucus was consistent across the surface (302±28 μm), possibly due to its ability to flow and re-distribute as a result of its water content. A similar result was observed by overlaying 1 μm red fluorescent beads onto the mucus layer (FIG. 3F). This in vitro generated mucus layer resembles the characteristic of in vivo inner colon mucus layer by forming a barrier competent to segregate the epithelium from luminal microbes and microbeads.⁴⁶ To visualize both the mucus and epithelium, the sample was dehydrated and the mucus was partially removed from the epithelium (dashed line is the boundary in FIG. 3G). E. coli were found only on the surface of the mucus layer and were not seen in contact with the epithelium. This result again demonstrates that the hydrated mucus layer formed a barrier to segregate epithelium and microbes (FIG. 3G).

The ALI culture method has been used for bronchial epithelial cells,⁴⁷ keratinocytes,⁴⁸ adenocarcinoma intestinal cell lines,³⁷ and intestinal organoids.⁴⁹ ALI has also been used to culture primary small intestinal epithelial cells on porous membrane inserts in a commercial product (MatTek's 3D tissue model from MatTek Corporation). However, MatTek's 3D tissue model has not been shown to support a dense, continuous, microbead or bacterial-separating mucus layer with controllable thickness. Applicant has tested primary human small intestinal (jejunum) epithelial cells using the above ALI strategies (DM or DM-VIP), but a dense and thick mucus layer was not generated. The disclosed methods are applicable to primary human colonic cells, and these data show for the first time the creation of an in vitro colonic mucus system possessing in-vivo-like mucus characteristics.

### Example 3

### A Mucus Layer Hinders the Effect of C. Difficile Toxin on Primary Human Colonic Epithelium

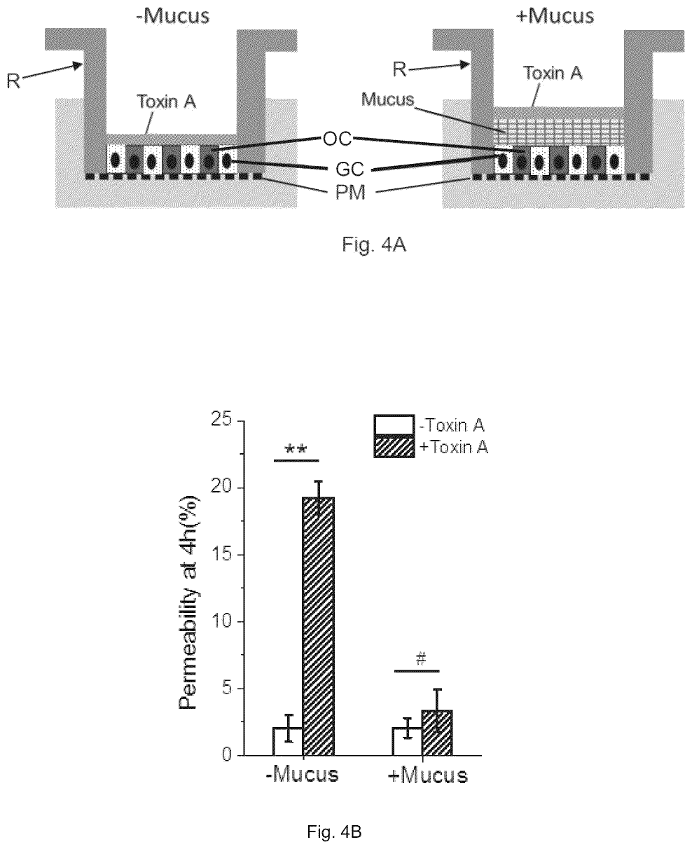

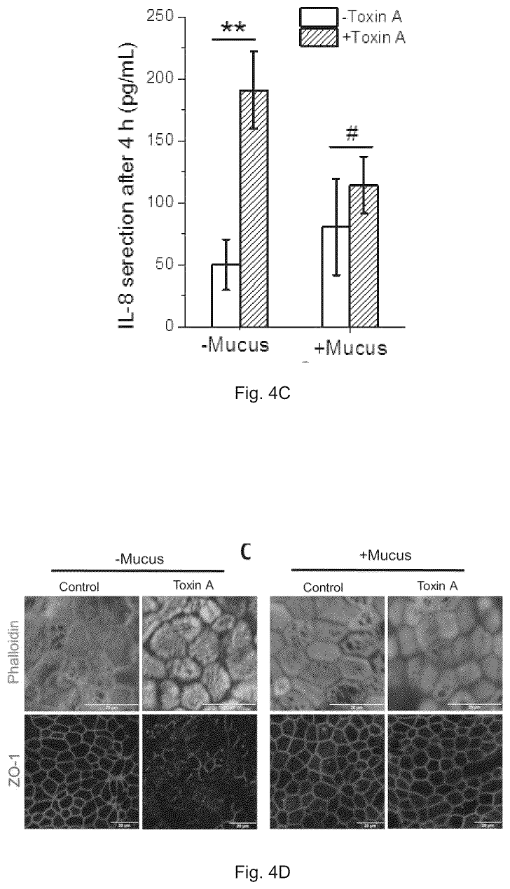

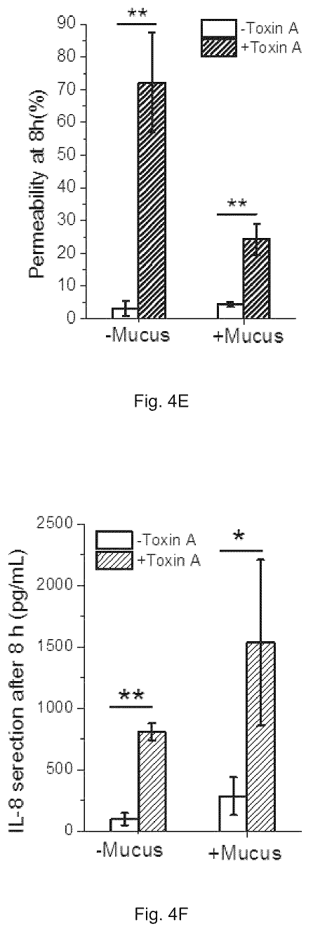

To demonstrate that the mucus layer improves the physiological relevance of an in vitro colonic epithelial model, primary human colonic epithelium (GC and/or OC) was exposed to C. difficile toxin in the absence or presence of a mucus layer (FIG. 4A). C. difficile produces two potent toxins, A and B, capable of inactivating host GTPases (including Rho, Rac, and Cdc42), leading to alteration of the epithelial barrier, damage to human intestinal mucosa and inflammation of the colon.⁵⁰ In the absence of a mucus layer in the submerged culture method, toxin A quickly induced cell damage, and the earliest change was observed within 2 h of toxin incubation. At 4 h, paracellular permeability was significantly increased in toxin-treated epithelial monolayers (FIG. 4B), and IL-8 secretion was also significantly enhanced (FIG. 4C). Both apical F-actin structures and ZO-1 tight junctions were markedly altered by toxin treatment. While control epithelial monolayers had organized F-actin in the apical brush border and the continuous “chicken wire” pattern of ZO-1, the toxin A treated monolayers exhibited cell rounding, disorganization and disruption of normal F-actin, and disassembly of ZO-1 architecture (FIG. 4D). This trend was observed at 8 h (FIGS. 4E and 4F) and 24 h. These results demonstrate that toxin A disrupts epithelial barrier function and elicits an immunological response, which is consistent with previous studies on other intestinal epithelium models.^{15, 51, 52}

In the presence of a mucus layer generated by ALI plus DM-VIP, cell responses to toxin treatment were not observed at 4 h in terms of change in permeability and IL-8 production (FIGS. 4B and 4C), as well as cell morphology, F-actin and ZO-1 architecture (FIG. 4D). However, changes in permeability and IL-8 production were observed at 8 h (FIGS. 4E and 4F). These results suggest that the mucus layer served as physical barrier or trap for toxins such that they reached the epithelium through pure diffusion (with or without mucus binding sites) and with convective mixing no longer able to accelerate toxin travel to the epithelium. These findings suggest our intestinal mucus-epithelium is an improved physiologically relevant model to study the host and pathogen factors for C. difficile infection.

### Example 4

### The Mucus Layer Serves as a Physical Barrier and Eliminates Bacteria-Induced Immune Response

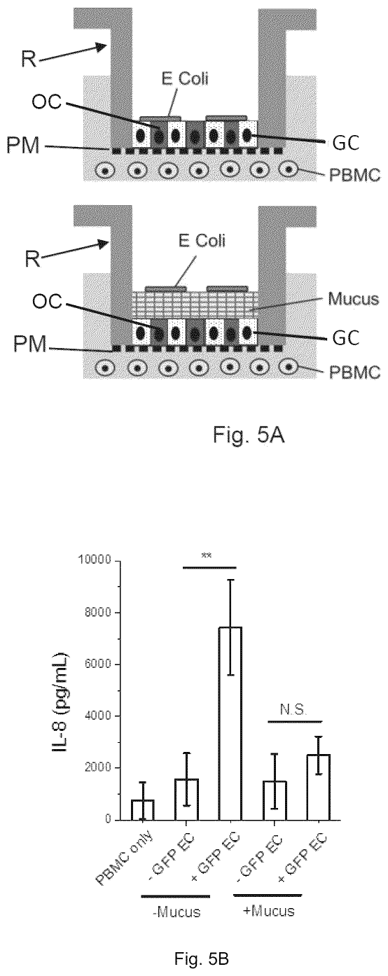

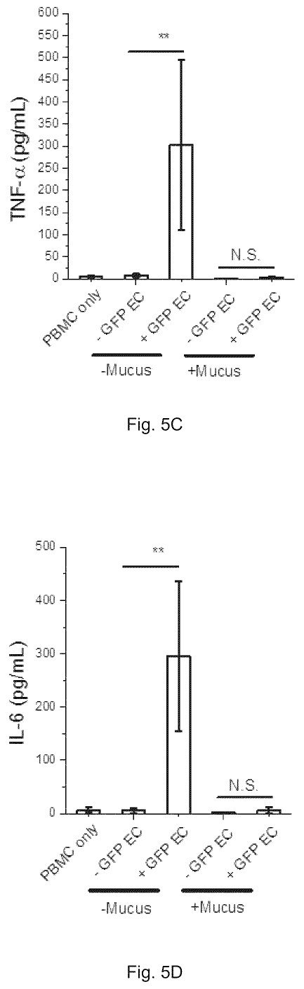

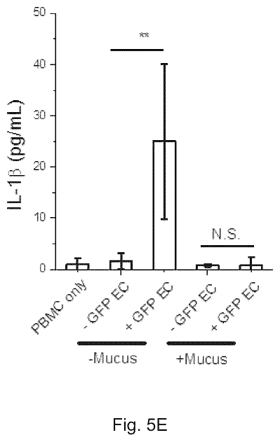

In vivo, the mucus layer acts as a vital component of the mucosal immune system by serving as a physical barrier to segregate commensal microbes from the host epithelium. Due to this physical segregation, commensal microbes coexist with epithelium without initiating a host inflammatory response.⁶ To demonstrate that the in vitro mucus layer could serve the same protective function, the epithelial monolayer was co-cultured with E. coli (luminal side) and peripheral blood mononuclear cells (PBMCs, basal side) for 24 h in the absence (cultured under the submerged method) or presence (generated by ALI plus DM-VIP method) of the mucus layer (FIG. 5A). Inflammatory cytokines secreted from the basal epithelial side were quantified (FIGS. 5B through 5E). Without exposure to E. coli, the PBMCs produced relatively low levels of cytokines. The co-culture of PBMCs and epithelium produced a similar level of cytokines. However, after the epithelium was exposed to the bacteria in the absence of the mucus layer, a significant increase in cytokine production was observed relative to that of the control without bacteria. These results are similar to that obtained when the non-mucus producing Caco-2 cells are grown in the presence of non-pathogenic E. coli.^{53,54}

In contrast, the cytokine response to co-cultured E. coli was eliminated in the presence of the mucus layer. Without challenge by E. coli, the co-culture of PBMCs, epithelium and mucus layer produced a relatively low level of cytokines. After a 24 h co-culture with E. coli, cytokine production were not statistically different from that without bacteria. These data demonstrate that the mucus layer successfully served to functionally segregate the microbes from the epithelium, thus eliminating the immune response and emulating the in vivo condition.

It will be understood that various details of the presently disclosed subject matter may be changed without departing from the scope of the presently disclosed subject matter. Furthermore, the foregoing description is for the purpose of illustration only, and not for the purpose of limitation.

### REFERENCES

All references listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Allen A, Carroll NJH. Adherent and Soluble Mucus in the Stomach and Duodenum. Digestive Diseases and Sciences 1985; 30: 55S.
2. Pelaseyed T, Bergström JH, Gustafsson JK, Ermund A, Birchenough GMH, Schütte A, van der Post S, Svensson F, Rodríguez-Piñeiro AM, Nyström EEL, Wising C, Johansson MEV, Hansson GC. The mucus and mucins of the goblet cells and enterocytes provide the first defense line of the gastrointestinal tract and interact with the immune system. Immunological reviews 2014; 260:8-20.
3. Murgia X, Loretz B, Hartwig O, Hittinger M, Lehr CM. The role of mucus on drug transport and its potential to affect therapeutic outcomes. Adv Drug Deliv Rev 2018; 124:82-97.
4. Lehr CM, Poelma FGJ, Junginger HE, Tukker JJ. An estimate of turnover time of intestinal mucus gel layer in the rat in situ loop. International Journal of Pharmaceutics 1991; 70:235-240.
5. Wei XC, Yang Z, Rey FE, Ridaura VK, Davidson NO, Gordon JI, Semenkovich CF. Fatty Acid Synthase Modulates Intestinal Barrier Function through Palmitoylation of Mucin 2. Cell Host & Microbe 2012; 11:140-152.
6. Johansson MEV, Phillipson M, Petersson J, Velcich A, Holm L, Hansson GC. The inner of the two Muc2 mucin-dependent mucus layers in colon is devoid of bacteria. Proceedings of the National Academy of Sciences of the United States of America 2008; 105:15064-15069.
7. Hansson GC. Role of mucus layers in gut infection and inflammation. Current Opinion in Microbiology 2012; 15:57-62.
8. Carlson TL, Lock JY, Carrier RL. Engineering the Mucus Barrier. Annual Review of Biomedical Engineering 2018; 20:197-220.
9. Werlang C, Cárcarmo-Oyarce G, Ribbeck K. Engineering mucus to study and influence the microbiome. Nature Reviews Materials 2019; 4:134-145.
10. Rogier EW, Frantz AL, Bruno MEC, Kaetzel CS. Secretory IgA is Concentrated in the Outer Layer of Colonic Mucus along with Gut Bacteria. Pathogens 2014; 3:390-403.
11. Gunasekara DB, Speer J, Wang Y, Nguyen DL, Reed MI, Smiddy NM, Parker JS, Fallon JK, Smith PC, Sims CE, Magness ST, Allbritton NL. A Monolayer of Primary Colonic Epithelium Generated on a Scaffold with a Gradient of Stiffness for Drug Transport Studies. Analytical Chemistry 2018; 90:13331-13340.
12. Quigley EMM. Gut bacteria in health and disease. Gastroenterology & hepatology 2013; 9:560-569.
13. Gagnon M, Berner AZ, Chervet N, Chassard C, Lacroix C. Comparison of the Caco-2, HT-29 and the mucus-secreting HT29-MTX intestinal cell models to investigate Salmonella adhesion and invasion. Journal of Microbiological Methods 2013; 94:274-279.
14. Lesuffleur T, Barbat A, Dussaulx E, Zweibaum A. Growth adaptation to methotrexate of HT-29 human colon carcinoma cells is associated with their ability to differentiate into columnar absorptive and mucus-secreting cells. Cancer Research 1990; 50:6334-6343.
15. Nusrat A, von Eichel-Streiber C, Turner JR, Verkade P, Madara JL, Parkos CA. Clostridium difficile Toxins Disrupt Epithelial Barrier Function by Altering Membrane Microdomain Localization of Tight Junction Proteins. Infection and Immunity 2001; 69:1329-1336.
16. Sato T, Vries RG, Snippert HJ, van de Wetering M, Barker N, Stange DE, van Es JH, Abo A, Kujala P, Peters PJ, Clevers H. Single Lgr5 stem cells build crypt-villus structures in vitro without a mesenchymal niche. Nature 2009; 459:262-U147.
17. Date S, Sato T. Mini-Gut Organoids: Reconstitution of Stem Cell Niche. In: Schekman R, ed. Annual Review of Cell and Developmental Biology, Vol 31. Volume 31, 2015:269-289.
18. Sato T, Stange DE, Ferrante M, Vries RGJ, van Es JH, van den Brink S, van Houdt WJ, Pronk A, van Gorp J, Siersema PD, Clevers H. Long-term Expansion of Epithelial Organoids From Human Colon, Adenoma, Adenocarcinoma, and Barrett's Epithelium. Gastroenterology 2011; 141:1762-1772.
19. Yin XL, Farin HF, van Es JH, Clevers H, Langer R, Karp JM. Niche-independent high-purity cultures of Lgr5(+) intestinal stem cells and their progeny. Nature Methods 2014; 11:106-112.
20. Fatehullah A, Appleton PL, Nathke IS. Cell and tissue polarity in the intestinal tract during tumourigenesis: cells still know the right way up, but tissue organization is lost. Philosophical Transactions of the Royal Society B-Biological Sciences 2013; 368:20130014.
21. Moon C, VanDussen KL, Miyoshi H, Stappenbeck TS. Development of a primary mouse intestinal epithelial cell monolayer culture system to evaluate factors that modulate IgA transcytosis. Mucosal Immunology 2014; 7:818-828.
22. VanDussen KL, Marinshaw JM, Shaikh N, Miyoshi H, Moon C, Tarr PI, Ciorba MA, Stappenbeck TS. Development of an enhanced human gastrointestinal epithelial culture system to facilitate patient-based assays. Gut 2015; 64:911-920.
23. In J, Foulke-Abel J, Zachos NC, Hansen A-M, Kaper JB, Bernstein HD, Halushka M, Blutt S, Estes MK, Donowitz M, Kovbasnjuk O. Enterohemorrhagic Escherichia coli Reduces Mucus and Intermicrovillar Bridges in Human Stem Cell-Derived Colonoids. Cellular and Molecular Gastroenterology and Hepatology 2016; 2:48-62.e3.
24. Kozuka K, He Y, Koo-Mccoy S, Kumaraswamy P, Nie B, Shaw K, Chan P, Leadbetter M, He L, Lewis JG, Zhong Z, Charmot D, Balaa M, King AJ, Caldwell JS, Siegel M. Development and Characterization of a Human and Mouse Intestinal Epithelial Cell Monolayer Platform. Stem Cell Reports 2017; 9:1976-1990.
25. Noel G, Baetz NW, Staab JF, Donowitz M, Kovbasnjuk O, Pasetti MF, Zachos NC. A primary human macrophage-enteroid co-culture model to investigate mucosal gut physiology and host-pathogen interactions. Scientific Reports 2017; 7:45270.
26. Puzan M, Hosic S, Ghio C, Koppes A. Enteric Nervous System Regulation of Intestinal Stem Cell Differentiation and Epithelial Monolayer Function. Scientific Reports 2018; 8:6313.
27. Wang YL, DiSalvo M, Gunasekara DB, Dutton J, Proctor A, Lebhar MS, Williamson IA, Speer J, Howard RL, Smiddy NM, Bultman SJ, Sims CE, Magness ST, Allbritton NL. Self-renewing Monolayer of Primary Colonic or Rectal Epithelial Cells. Cellular and Molecular Gastroenterology and Hepatology 2017; 4:165-+.
28. Madden LR, Nguyen TV, Garcia-Mojica S, Shah V, Le AV, Peier A, Visconti R, Parker EM, Presnell SC, Nguyen DG, Retting KN. Bioprinted 3D Primary Human Intestinal Tissues Model Aspects of Native Physiology and ADME/Tox Functions. iScience 2018; 2:156-167.
29. Wang Y, Kim R, Hwang S-HJ, Dutton J, Sims CE, Allbritton NL. Analysis of Interleukin 8 Secretion by a Stem-Cell-Derived Human-Intestinal-Epithelial-Monolayer Platform. Analytical Chemistry 2018; 90:11523-11530.
30. Wang YL, Kim R, Gunasekara DB, Reed MI, DiSalvo M, Nguyen DL, Bultman SJ, Sims CE, Magness ST, Allbritton NL. Formation of Human Colonic Crypt Array by Application of Chemical Gradients Across a Shaped Epithelial Monolayer. Cellular and Molecular Gastroenterology and Hepatology 2018; 5:113-130.
31. Whitcutt MJ, Adler KB, Wu R. A biphasic chamber system for maintaining polarity of differentiation of cultured respiratory tract epithelial cells. In Vitro Cellular & Developmental Biology 1988; 24:420-428.
32. Gray TE, Guzman K, Davis CW, Abdullah LH, Nettesheim P. Mucociliary differentiation of serially passaged normal human tracheobronchial epithelial cells. American Journal of Respiratory Cell and Molecular Biology 1996; 14:104-112.
33. Raredon MSB, Ghaedi M, Calle EA, Niklason LE. A Rotating Bioreactor for Scalable Culture and Differentiation of Respiratory Epithelium. Cell Medicine 2015; 7:109-121.
34. O′Boyle N, Sutherland E, Berry CC, Davies RL. Temporal dynamics of ovine airway epithelial cell differentiation at an air-liquid interface. Plos One 2017; 12.
35. Ootani A, Toda S, Fujimoto K, Sugihara H. An air-liquid interface promotes the differentiation of gastric surface mucous cells (GSM06) in culture. Biochemical and Biophysical Research Communications 2000; 271:741-746.
36. Yokoyama F, Sakata Y, Ootani A, Fujise T, Kakimoto T, Amemori S, Shiraishi R, Kuroki T, Tsunada S, Iwakiri R, Fujimoto K. Differentiation of gastric surface mucous cells (GSM06) induced by air-liquid interface is regulated partly through mitogen-activated protein kinase pathway. Journal of Gastroenterology and Hepatology 2007; 22:2310-2315.
37. Navabi N, McGuckin MA, Linden SK. Gastrointestinal Cell Lines Form Polarized Epithelia with an Adherent Mucus Layer when Cultured in Semi-Wet Interfaces with Mechanical Stimulation. Plos One 2013; 8.
38. Elkins MR, Bye PTP. Mechanisms and applications of hypertonic saline. Journal of the Royal Society of Medicine 2011; 104:S2-S5.
39. J. T, J.M. P, V.P. K, X.D. Z. Effect of osmotic response element binding protein on mucus secretion with hypertonicity in human airway epithelial cells. Zhonghua Yi Xue Za Zhi 2011; 91:549-553.
40. Ludeking A, Fegert P, Blin N, Gott P. Osmotic changes and ethanol modify TFF gene expression in gastrointestinal cell lines. Febs Letters 1998; 439:180-184.
41. Shields R, Miles JB, Gilbertson C. Absorption and secretion of water and electrolytes by the intact colon in a patient with primary aldosteronism. British Medical Journal 1968; 1:93-96.
42. Wapnir RA, Teichberg S. Regulation mechanisms of intestinal secretion: implications in nutrient absorption. The Journal of Nutritional Biochemistry 2002; 13:190-199.
43. Koch TR, Michener SR, Go VLW. Plasma vasoactive intestinal polypeptide concentration determination in patients with diarrhea. Gastroenterology 1991; 100:99-106.
44. Schwartz CJ, Kimberg DV, Sheerin HE, Field M, Said SI. Vasoactive intestinal peptide stimulation of adenylate cyclase and active electrolyte secretion in intestinal mucosa. Journal of Clinical Investigation 1974; 54:536-544.
45. Wu X, Conlin VS, Morampudi V, Ryz NR, Nasser Y, Bhinder G, Bergstrom KS, Yu HB, Waterhouse CCM, Buchan AMJ, Popescu OE, Gibson WT, Waschek JA, Vallance BA, Jacobson K. Vasoactive Intestinal Polypeptide Promotes Intestinal Barrier Homeostasis and Protection Against Colitis in Mice. Plos One 2015; 10: e0125225.
46. Johansson MEV, Gustafsson JK, Holmén-Larsson J, Jabbar KS, Xia L, Xu H, Ghishan FK, Carvalho FA, Gewirtz AT, Sjövall H, Hansson GC. Bacteria penetrate the normally impenetrable inner colon mucus layer in both murine colitis models and patients with ulcerative colitis. Gut 2014; 63:281-291.
47. Lin HX, Li H, Cho HJ, Bian S, Roh HJ, Lee MK, Kim JS, Chung SJ, Shim CK, Kim DD. Air-liquid interface (ALI) culture of human bronchial epithelial cell monolayers as an in vitro model for airway drug transport studies. Journal of Pharmaceutical Sciences 2007; 96:341-350.
48. Bernstam LI, Vaughan FL, Bernstein IA. Keratinocytes grown at the air-liquid interface. In Vitro Cellular & Developmental Biology 1986; 22:695-705.
49. Ootani A, Li XN, Sangiorgi E, Ho QT, Ueno H, Toda S, Sugihara H, Fujimoto K, Weissman IL, Capecchi MR, Kuo CJ. Sustained in vitro intestinal epithelial culture within a Wnt-dependent stem cell niche. Nature Medicine 2009; 15:1-U140.
50. Voth DE, Ballard JD. Clostridium difficile toxins: mechanism of action and role in disease. Clinical microbiology reviews 2005; 18:247-263.
51. He D, Sougioultzis S, Hagen S, Liu J, Keates S, Keates AC, Pothoulakis C, LaMont JT. Clostridium difficile toxin A triggers human colonocyte IL-8 release via mitochondrial oxygen radical generation. Gastroenterology 2002; 122:1048-1057.
52. Mahida YR, Makh S, Hyde S, Gray T, Borriello SP. Effect of Clostridium difficile toxin A on human intestinal epithelial cells: induction of interleukin 8 production and apoptosis after cell detachment. Gut 1996; 38:337-347.
53. Haller D, Bode C, Hammes WP, Pfeifer AMA, Schiffrin EJ, Blum S. Non-pathogenic bacteria elicit a differential cytokine response by intestinal epithelial cell/leucocyte co-cultures. Gut 2000; 47:79-87.
54. Parlesak A, Haller D, Brinz S, Baeuerlein A, Bode C. Modulation of Cytokine Release by Differentiated CACO-2 Cells in a Compartmentalized Coculture Model with Mononuclear Leucocytes and Nonpathogenic Bacteria. Scandinavian Journal of Immunology 2004; 60:477-485.

1-15. (canceled)

16. A live cell construct comprising:
(a) a basal reservoir comprising a first liquid medium;
(b) a luminal reservoir held within a basal well, the luminal reservoir comprising:
(i) a surface comprising a porous membrane; and
(ii) a cell monolayer cultured on the surface and comprising a basal side and a luminal side, the basal side comprising mucus producing cells that secrete a mucus layer on the luminal side of the cell monolayer; and
(iii) an air-liquid interface at the luminal side of the cell monolayer.

17. The live cell construct of claim 16, wherein the mucus layer comprises a basal side and a luminal side, wherein the basal side is below and adjacent to the mucus producing cells.

18. The live cell construct of claim 16, wherein the mucus layer is substantially impenetrable to micro objects.

19. The live cell construct of claim 16, wherein the thickness of the mucus layer is about 1 micron to about 1 cm.

20. The live cell construct of claim 18, wherein the mucus layer is impenetrable to micro-objects in a size range from about 1 micron to about 100 microns.

21. (canceled)

22. A method of determining the ability of an organism, a drug, or a particle to traverse a mucus layer of a cell monolayer, the method comprising:
contacting the luminal side of the mucus layer of the live cell construct of claim 16 with the organism, drug, or particle; and
measuring the distance that the organism, drug, or particle moves into the mucus layer, thereby determining the ability of the organism, drug or particle to traverse the mucus layer of the cell monolayer of the live cell construct.

23. The method of claim 22, wherein the organism is a bacterium, a virus, a fungus, protozoan, and/or a helminth.

24. The live cell construct of claim 16, wherein the basal reservoir is present below the basal side of the cell monolayer and the luminal reservoir is present above the luminal side of the cell monolayer, and removing the first liquid medium produces the air-liquid interface at the luminal side of the cell monolayer.

25. The live cell construct of claim 16, further comprising a physical barrier on or above the luminal side of the cell monolayer, wherein the physical barrier is (i) completely or substantially impermeable to liquid and mucin, or (ii) permeable to water but impermeable or substantially impermeable to mucin

26. The live cell construct of claim 25, wherein the physical barrier is in direct contact with the luminal side of the cell monolayer and/or the mucus layer.

27. The live cell construct of claim 25, wherein the depth of the first liquid medium is in a range of about 0.001 mm to about 10 mm, and the first liquid medium is between the physical barrier and the luminal side of the cell monolayer.

28. The live cell construct of claim 16, wherein the first liquid medium further comprises a hormone, a chemical additive, a food additive, a bacterial metabolite, and/or a physiologically hypertonic salt solution, wherein the chemical additive is selected from the group consisting of: butyrate, bone morphogenetic protein (BMP), dibenzazepine, DAPT, LY411575, forskolin, guaifenesin, carbachol, prostaglandins, phorbol 12-myristate 13-acetate, histamine, and N-(1-oxobutyl)-cyclic 3′, 5′-(hydrogen phosphate) 2′-butanoate-adenosine monosodium salt.

29. The live cell construct of claim 16, wherein the basal reservoir is present below the basal side of the cell monolayer and a luminal reservoir is present above the luminal side of the cell monolayer, and wherein the volume of a liquid medium in the luminal reservoir is adjusted to a depth in a range of about 0.001 mm to about 10 mm above the luminal surface of the cell monolayer.
